(19) United States
(12) Patent Application Publication
(10) Pub. No.: US 2021/0196337 A1
(43) Pub. Date: Jul. 1, 2021

(54) METHOD FOR TREATMENT FOR COMBINATION COLD ATMOSPHERIC PLASMA THERAPY OF SOLID TUMORS

(71) Applicant: Jerome Canady Research Institute for Advanced Biological and Technological Sciences, Takoma Park, MD (US)

(72) Inventors: Jerome Canady, Lakeland, FL (US); Xiaoqian Cheng, Fairfax, VA (US); Saravana Murthy, Owings Mills, MD (US); Taisen Zhuang, Rockvillle, MD (US); Lawan Ly, Annandale, VA (US); Olivia Jones, Takoma Park, MD (US)

(21) Appl. No.: 17/189,185

(22) Filed: Mar. 1, 2021

Related U.S. Application Data
(63) Continuation-in-part of application No. 17/135,866, filed on Dec. 28, 2020.
(60) Provisional application No. 62/983,636, filed on Feb. 29, 2020.
(60) Provisional application No. 62/953,783, filed on Dec. 26, 2019.

(51) Int. Cl. A61B 18/00

(52) U.S. Cl. CPC A61B 18/00; A61B 2018/00744; A61B 2018/00583

(57) ABSTRACT

A method for treatment of solid cancer tumors. The method includes pre-operatively treating a patient having a solid cancer tumor with at least one of chemotherapy and radiation therapy, surgically removing the solid cancer tumor from the patient, applying cold atmospheric plasma to the surgical margins surrounding the area in the patient from which the solid cancer tumor was removed, performing at least one of intra-operative chemotherapy or radiation therapy, and post-operatively performing at least one of chemotherapy and radiation therapy on the patient.

### CROSS-REFERENCE TO RELATED APPLICATIONS

The present application claims the benefit of the filing date of U.S. Provisional Patent Application Ser. No. 62/983,636 filed by the present inventors on Feb. 29, 2020. The present application further is a continuation-in-part of U.S. Non-provisional patent application Ser. No. 17/135,866, filed on Dec. 28, 2020, which claims the benefit of the filing of U.S. Provisional Patent Application Ser. No. 62/953,783 filed by the present inventors on Dec. 26, 2019.

The aforementioned patent applications are hereby incorporated by reference in their entirety.

### STATEMENT REGARDING FEDERALLY SPONSORED RESEARCH OR DEVELOPMENT

None.

### BACKGROUND OF THE INVENTION

### Field of the Invention

The present invention relates to systems and methods for treating cancer with cold atmospheric plasma.

### Brief Description of the Related Art

Cancer is a disease that results from abnormal cells growing without control. Normal cells are pre-controlled by genes that promote the process of cell division. Life is dependent on the function of cells to reproduce or make exact copies of each other. Cell division is a process that one cell divides into two cells. This process is called mitosis. The cells go through a cell cycle, a process of growing, breaking down and repair about 50 times before death. When the normal cell cannot be repaired, it goes through a pre-programmed cell death or apoptosis. Cancer cells avoid repair and apoptosis pathway and continue grow rapidly, uncontrolled that results in tumor formation. In summary cancer is a disease of mitosis. The normal regulatory pathways for mitosis are ignored or bypass by cancer cells. Cancer starts when a single cell is transformed or converted from normal cell to a cancer cell. This process is a result from change in function of genes that control growth or suppress tumor formation.

Cancer is among leading causes of death worldwide. In descending order breast cancer, lung and bronchus cancer, prostate cancer, colon and rectum, anal, melanoma of the skin, bladder cancer, non-Hodgkin Lymphoma, kidney and renal and pelvis cancer, endometrial cancer, leukemia, pancreatic cancer, thyroid cancer and liver are the most common new cancer cases in 2018.

Breast cancer is the most common cancer diagnosed among US women and is the second leading cause of death among women after lung cancer. The estimated cases and deaths in 2017 related to breast cancer are 252,710 new cases of invasive breast cancer and 63,410 cases of in situ breast cancer and 40,000 deaths. Cody and Van Zee³ reported that 180,000 women underwent breast—conserving surgery. The other breast cancer epidemic reported by Cody and Van Zee that the re-excision rate for microscopic positive margins at the surgical site range from 10% to 50%.

Tumor spreading within the abdominal cavity is defined as peritoneal carcinomatosis (PC). Tumor arises primarily from peritoneal surface or visceral organs. Uncontrolled expansion of the primary tumor leads to spreading that allows the tumor cells to peel off and circulate within the peritoneal fluid, permitting an increase progression of disease given the decrease growth inhibition in the implanted peritoneal metastases. The end result of this diffuse tumor cell implantation of vital organs is malnutrition, bowel obstruction and death.

Surgical resection has not been accepted as a standard option for PC from GI cancers. Median survival is 6 to 12 months with systemic chemotherapy. In the 1990's Dr. Paul Sugarbaker and others published cytoreductive surgery (CRS), peritonectomy combined with hyperthermic intraperitoneal chemotherapy (HIPEC) to treat peritoneal surface malignancy. Several authors have reported that CRS plus HIPEC have increased survival in selected patients with colorectal, appendiceal and primary peritoneal cases. The application of CRS and HIPEC is an aggressive local regional approach for the treatment PC. The purpose of CRS is to leave little or no residual disease within the abdomen. After surgery HIPEC is used to wash the abdominal cavity with a heated high concentrated chemo-therapeutic drug. The pharmokinetic benefits of the intraperitoneal route of chemotherapy are increased drug concentrations, locally dose intensive therapy and the synergistic effect of hyperthermia. Hyperthermia alone has a cytotoxic effect on malignant cells and facilitate greater tissue penetration of the antineoplastic agents. The combination of CRS and HIPEC is believed to achieve macroscopic and microscopic disease clearance and possible improved survival. Despite the promise of improved survival in patients with PC, HIPEC is associated with significant morbidity and mortality.

Lympho-proliferative cancers (leukemia, lymphoma) most patients diagnosed with solid cancerous tumors (i.e., colon, ovarian, cervical, lung, liver, bile duct, brain, bone, small intestine, melanoma, sarcoma) require surgical treatment as a single modality therapy or as a major component in a multi-modality therapy. The main limitations of surgery are the inability of the surgeon to identify occult disease and unable to completely resect tumors adjacent to vital organs. As a result, the tumor returns in the form of loco-regional or metachronous distant metastasis. Application of chemotherapy or radiation therapy before, during- or after surgery may decrease disease recurrence.

Cold atmospheric plasma (CAP) has been extensively studied in various biomedical fields. It is a novel approach to targeted cancer treatment and has demonstrated its anti-cancer effects in vitro. See, Rowe, W., X. Cheng, L. Ly, et al., The Canady Helios cold plasma scalpel significantly decreases viability in malignant solid tumor cells in a dose-dependent manner. Plasma, 2018. 1(1): p. 177-188; Barekzi, N. and M. Laroussi, Effects of low temperature plasmas on cancer cells. Plasma Processes and Polymers, 2013. 10(12): p. 1039-1050; Barekzi, N. und M. Laroussi, Dose-dependent killing or leukemia cells by low temperature plasma. Journal of Physics D: Applied Physics, 2012.45(42); and Keidar, M., R. Walk, A. Shashurin; et al., Cold plasma selectivity and the possibility of a paradigm shift in cancer therapy. Br J Cancer, 2011. 105(9): p. 1295-301. The detailed mechanism has not been fully elucidated; however, studies have established that CAP selectively induces apoptosis and DNA damage in tumor cells. Arndt, S., M. Landthaler, J. L. Zimmermann, et al., Effects of cold atmospheric plasma (cap) on ss-defensins, inflammatory cytokines, and apoptosis-related molecules in keratinocytes in vitro and in vivo. PLoS One, 2015. 10(3): p. e0120041; Bauer, G., D. Sersenova, D. B. Graves, et al., Cold atmospheric plasma and plasma activated medium trigger rans-based tumor cell apoptosis. Sci Rep, 2019. 9(1): p. 14210; Cheng, X., W. Rowe, L. Ly, et al., Treatment of triple-negative breast cancer cells with the Canady cold plasma conversion system: Preliminary results. Plasma, 2018. 1 (1): p. 218-228. Further research indicates low doses of CAP does not damage normal tissue. See, e.g., Lee, J. II., J. Y. Om, Y. H. Kim, et al., Selective killing effects of cold atmospheric pressure plasma with no induced dysfunction of epidermal growth factor receptor in oral squamous cell carcinoma. PLoS One, 2016. 11(2): p. e0150279. Recently, indirect CAP treatment was effective for the treatment of CCA in vitro, selectively killing CCA cells over normal hepatocytes. Vaquero, J., F. Judee, M. Vallette, et al., Cold-atmospheric plasma induces tumor cell death in preclinical in vivo and in vitro models of human cholangiocarcinoma. Cancers, 2020. 12(5). Research on CAP in combination with other therapies has shown some potential synergism with anti-neoplastic agents in melanoma cells (Sagwal, S. K., G. Pasqual-Melo, Y. Bodnar, et al., Combination of chemotherapy and physical plasma elicits melanoma cell death via upregulation of slc22al 6. Cell Death Dis, 2018. 9(12): p. 1179), drug loaded nanoparticles in breast cancer cells (Zhu, W., S. J. Lee, N.J. Castro, et al., Synergistic effect of cold atmospheric plasma and drug loaded core-shell nanoparticles on inhibiting breast cancer cell growth. Sci Rep, 2016.6: p. 21974), and gemcitabine in murine pancreatic cancer cells (Masur, K., M. van Behr, S. Bekeschus, et al., Synergistic inhibition of tumor cell proliferation by cold plasma and gemcitabine. Plasma Processes and Polymers, 2015.12(12): p. 1377-1382).

Delivery of cold atmospheric plasma at the surgical margins immediately after tumor resection has shown potential as an anti-cancer therapy. A Canady Cold Plasma Conversion System is an electrosurgical system that produces CAP for the treatment of surgical margins upon tumor resection (U.S. Pat. No. 9,999,462). One of the advantages of cold atmospheric plasma systems is that the CAP temperature remains between 26-30° C. during the duration of the treatment (Cheng, X., et al., Treatment of Triple-Negative Breast Cancer Cells with the Canady Cold Plasma Conversion System: Preliminary Results. Plasma, 2018. 1(1): p. 218-228) and does not cause any thermal or physical damage to normal tissue (Ly, L., et al., A New Cold Plasma Jet: Performance Evaluation of Cold Plasma, Hybrid Plasma and Argon Plasma Coagulation. Plasma, 2018. 1(1): p. 189-200).

### SUMMARY OF THE INVENTION

In a preferred embodiment, the present invention is a method for treatment of solid cancer tumors. The method comprises pre-operatively treating a patient having a solid cancer tumor with at least one of chemotherapy and radiation therapy, surgically removing the solid cancer tumor, and applying cold atmospheric plasma to the surgical margins surrounding the area in the patient from which the solid cancer tumor was removed. The step of pre-operatively treating a patient may comprise treating the patient pre-operatively with both chemotherapy and radiation therapy. The method may further comprise post-operatively treating the patient having a solid cancer tumor with at least one of chemotherapy and radiation therapy. The step of applying cold atmospheric plasma to the surgical margins comprises applying cold atmospheric plasma to the surgical margins for no more than 5 minutes, no more than 3 minutes or no more than one minute. Further, the step of applying cold atmospheric plasma to the surgical margins may comprise applying cold atmospheric plasma to the surgical margins at 120p with a helium flow rate of 3 l/min. Still further, the method may comprise intra-operatively treating a patient having a solid cancer tumor with at least one of chemotherapy and radiation therapy. The method further may comprise performing at least one of cytoreductive surgery and peritonectomy combined with hyperthermic intraperitoneal chemotherapy on the patient.

Still other aspects, features, and advantages of the present invention are readily apparent from the following detailed description, simply by illustrating a preferable embodiments and implementations. The present invention is also capable of other and different embodiments and its several details can be modified in various obvious respects, all without departing from the spirit and scope of the present invention. Accordingly, the drawings and descriptions are to be regarded as illustrative in nature, and not as restrictive. Additional objects and advantages of the invention will be set forth in part in the description which follows and in part will be obvious from the description or may be learned by practice of the invention.

### BRIEF DESCRIPTION OF THE DRAWINGS

For a more complete understanding of the present invention and the advantages thereof, reference is now made to the following description and the accompanying drawings, in which:

FIG. 1 is a flow chart illustrating a method for treatment of solid tumors with a combination of CAP and other therapies in accordance with a preferred embodiment of the present invention.

FIG. 2 is a perspective view of a preferred embodiment of a gas-enhanced electrosurgical generator that may be used in a preferred embodiment of the present invention.

FIG. 3 is a block diagram of a cold atmospheric plasma generator in accordance with a preferred embodiment of the present invention.

FIG. 4A is a block diagram of an embodiment of a cold atmospheric plasma system with an electrosurgical generator and a low frequency converter for producing cold plasma.

FIG. 4B is a block diagram of an embodiment of an integrated cold atmospheric plasma system that can perform multiple types of plasma surgeries.

FIG. 5 is perspective view of a cold atmospheric plasma probe that may be used in a preferred embodiment of the present invention.

FIG. 6A is an assembly view of a handpiece of a cold atmospheric plasma probe that may be used in a preferred embodiment of the present invention.

FIG. 6B is an assembly view of a cable harness of a cold atmospheric plasma probe that may be used in a preferred embodiment of the present invention.

FIGS. 7A-7B are tables summarizing treatments performed on patients in accordance with embodiments of the present invention.

FIGS. 8A-8C are graphs illustrating intraoperative data of O2 saturation, body temperature and pulse of Subject 1 in FIGS. 7A-7B.

FIGS. 9A-9C are graphs illustrating intraoperative data of O2 saturation, body temperature and pulse of Subject 2 in FIGS. 7A-7B.

FIGS. 10A-10C are graphs illustrating intraoperative data of O2 saturation, body temperature and pulse of Subject 3 in FIGS. 7A-7B.

FIGS. 11A-11C are graphs illustrating intraoperative data of O2 saturation, body temperature and pulse of Subject 4 in FIGS. 7A-7B.

FIGS. 12A-12C are graphs illustrating intraoperative data of O2 saturation, body temperature and pulse of Subject 5 in FIGS. 7A-7B.

FIGS. 13A-13C are graphs illustrating intraoperative data of O2 saturation, body temperature and pulse of Subject 6 in FIGS. 7A-7B.

FIGS. 14A-14C are graphs illustrating intraoperative data of O2 saturation, body temperature and pulse of Subject 7 in FIGS. 7A-7B.

FIGS. 15A-15C are graphs illustrating intraoperative data of O2 saturation, body temperature and pulse of Subject 8 in FIGS. 7A-7B.

FIGS. 16A-16C are graphs illustrating intraoperative data of O2 saturation, body temperature and pulse of Subject 9 in FIGS. 7A-7B.

FIGS. 17A-17C are graphs illustrating intraoperative data of O2 saturation, body temperature and pulse of Subject 10 in FIGS. 7A-7B.

FIGS. 18A-18C are graphs illustrating intraoperative data of O2 saturation, body temperature and pulse of Subject 11 in FIGS. 7A-7B.

FIGS. 19A-19C are graphs illustrating intraoperative data of O2 saturation, body temperature and pulse of Subject 12 in FIGS. 7A-7B.

FIGS. 20A-20C are graphs illustrating intraoperative data of O2 saturation, body temperature and pulse of Subject 13 in FIGS. 7A-7B.

FIGS. 21A-21C are graphs illustrating intraoperative data of O2 saturation, body temperature and pulse of Subject 14 in FIGS. 7A-7B.

FIGS. 22A-22C are graphs illustrating intraoperative data of O2 saturation, body temperature and pulse of Subject 15 in FIGS. 7A-7B.

FIG. 23 is a set of culture images of treated and untreated cancer cells from Subject 6 (R0002), who was treated for myxofibrosarcoma.

FIG. 24 is a set of culture images of treated and untreated cancer cells from Subject 10 (R0007), who was treated for cholangiocarcinoma.

FIG. 25 is a set of culture images of treated and untreated cancer cells from Subject 12 (R0009), who was treated for pleomorphic sarcoma.

FIG. 26 is a set of culture images of treated and untreated cancer cells from Subject 13 (R0011), who was treated for Chordoma.

FIG. 27 is a set of culture images of treated and untreated cancer cells from Subject 15 (R0012), who was treated for pleomorphic spindle cell sarcoma.

FIGS. 28A-C are graphs illustrating the synergistic effect between CAP one and FOLFIRINOX.

FIG. 29 is a diagram showing translational molecular processing of blood samples taken from the subject patients in accordance with a preferred embodiment of the present invention.

FIG. 30 is a diagram showing translational molecular processing of tissue samples taken from the subject patients in accordance with a preferred embodiment of the present invention.

FIGS. 31A-31E illustrate a method for gene expression profiling in accordance with a preferred embodiment of the present invention.

FIGS. 32A-32B illustrate a method for protein profiling in accordance with a preferred embodiment of the present invention.

FIGS. 32A-32B illustrate a method for protein profiling in accordance with a preferred embodiment of the present invention.

FIG. 33 illustrates a method for exosome profiling in accordance with a preferred embodiment of the present invention.

FIGS. 34A-34B illustrate a method for Human T Lymphocyte Isolation in accordance with a preferred embodiment of the present invention.

FIGS. 35A-35B illustrate a method for Primary cell culture and immortalization in accordance with a preferred embodiment of the present invention.

FIG. 36 illustrates a method for a cancer stem cell (CSC) culture in accordance with a preferred embodiment of the present invention.

FIG. 37 illustrates a method for a Primary cancer cell culture in accordance with a preferred embodiment of the present invention.

FIG. 38 illustrates a method for primary cell immortalization (with human telomerase reverse transcriptase hTERT or SV40 T antigen) in accordance with a preferred embodiment of the present invention.

FIGS. 39A-39D illustrate a method for an organoid culture in accordance with a preferred embodiment of the present invention.

FIG. 40 illustrates a method for a gene expression profiling in accordance with a preferred embodiment of the present invention.

FIG. 41 illustrates a method for a protein profiling in accordance with a preferred embodiment of the present invention.

### DETAILED DESCRIPTION OF THE PREFERRED EMBODIMENTS

Cholangiocarcinomas are rare with a low five-year survival rate. Cold atmospheric plasma (CAP) is a promising technology as a selective cancer treatment due to its anticancer properties. In pancreatic and liver cancers, FOLFIRINOX has emerged as an effective combination cancer drug treatment. It has been reported that FOLFIRINOX increased overall patient survival over gemcitabine treatment in patients with metastatic pancreatic cancer but is limited due to toxicity. See, T. Conroy et al., N. Engl. J. Med., 364, pp. 1817-1825 (2011) and T. Conroy et al., N. Engl. J. Med., 379, pp. 2395-2406 (2018).

The present invention is a method for treating cancer with a combination of cold atmospheric plasma (CAP) in combination with other therapies. CAP has been shown to produce no thermal damage to healthy tissues after the margins of resected tumor tissues. Rossi F, De Mitri R, Bobin S, et al. Plasma sterilisation: mechanisms overview and influence of discharge parameters. In: D'Agostino R, Favia P, Oehr C, et al., editors. (eds) Plasma processes and polymers. Germany: Wiley-VCH Verlag GmbH & Co. KGaA, 2005, pp. 319-331. CAP also has been shown to have an effect on cancer tissues and cells located at a remote site form the treated lesion. C, Berganza C, Zhang J. Cold atmospheric plasma: methods of production and application in dentistry and oncology. Med Gas Res 2013; 3: 21.

The present invention uses CAP in patients undergoing surgical treatment of solid tumors in combination with systemic chemotherapy, hyperthermic intraoperative chemotherapy, intraoperative or external beam radiation therapy or cytoreductive surgery for the treatment of solid tumors. The method is to apply CAP treatment for ablation of cancerous cells at the surgical margin and macroscopic tumor sites in combination with systemic chemotherapy, cytoreductive surgery, HIPEC, external beam or intraoperative radiation therapy for solid tumors.

Cold atmospheric plasma (CAP) provides a unique, rich environment of reactive oxygen species (ROS), reactive nitrogen species (RNS), charged particles, photons, and electric field. This unique combination of chemical and physical properties of CAP technology has enabled recent biomedical applications including cancer therapy. Some chemical components of the CAP are highly selective, such as ROS, which might promote a “plasma killing effect,” while others such as RNS could produce a “plasma healing” effect. Combining these species in various controlled blends provides an unprecedented potential to activate specific signaling pathways in cells. Combining RON species in various controlled blends provides an unprecedented potential to activate specific signaling pathways in cells.

What makes plasma absolutely unique is its ability to self-organize and form coherent structures. These coherent structures modulate plasma chemistry and composition, including reactive species, the electric field and charged particles. These coherent structures tend to modulate plasma chemistry and composition, including reactive species, the electric field and charged particles. Formation of coherent plasma structures allows the plasma to adapt to external boundary conditions, such as different cells types and their contextual tissues. As result, plasma interaction with cells is altered leading to differential effect of plasma on different cells, i.e., selective killing of cancer cells in comparison with normal cells.

The mechanism by which CAP affects the cancer is based on the production and efficient delivery of reactive oxygen and nitrogen species (RONS) analogous to those produced naturally by cells. The CAP-originated reactive species will cause a noticeable rise of intracellular ROS, which weakens the intracellular antioxidant system and further causes serious DNA double-strand break. As a result, cell cycle arrest and apoptosis based on mitochondrion-pathway or tumor necrosis factor receptor-pathway occur.

CAP is capable of gentle non-thermal modification of the radical balance in cells leading to apoptosis rather than necrosis. In contrast, conventional lasers in medical devices are based on the thermal interaction with tissues, which lead to necrosis and permanent tissue damage. Tuning the gas pressure (helium), gas composition (helium/oxygen) and energy can control the chemical responses of the tissue to a CAP jet. Various chemical components of the CAP jet (e.g., RONS) are highly selective, such as oxygen, which might promote a “plasma killing effect”, while nitric oxide could produce a “plasma healing” effect. Conceptually, it is the introduction/delivery of these potentially selective toxic ion-species into the tumors through diffusion processes and the electric field produced by plasma that will form this foundation of new cancer therapy. Recently our group and other groups world-wide reported that the CAP jet; a) selectively kills cancer cells in vitro with a significantly lesser effect on normal cells; and b) significantly reduces tumor size in vivo. We established the following mechanism to describe the plasma effect: the synergistic effect of selective diffusion of RONS into tumor cells (via activation of the aquiporins (AQP)) and the high basic RONS level in tumor cells would make a dramatic difference in the response of normal and tumor cells to CAP, thereby crossing a survival threshold for tumor cells and leading to cell death through DNA damage, apoptosis or cell cycle arrest.

In addition, the RONS metabolism and oxidative stress-responsive genes are deregulated following CAP application, and the differential effects of CAP on various cancer cells results in G2/M arrest. Fewer normal cells were in S-phase (˜10%) compared to the two cancer cell lines (transformed cells are highly proliferative): ˜50% for 308 cells and ˜45% for PAM212 cells. No increase in the fraction of cells in the S-phase after CAP treatment was observed for the three cell types: their number either remained the same or decreased. However, the present inventors observed an increase in the ranging of standard deviation value of CAP treated cells in S-phase of around ˜20% for all three cell types suggesting that not all cells within the population of cells responded the same to CAP treatment. While there is no significant difference in the numbers of cells in the S-phase of the cell cycle, one can see that the number of cells in the G2/M fraction increased by ˜25% for normal cells and two- to threefold for transformed cells. The increase in the fraction of cells at the G2/M-phase of the cell cycle is accompanied a decrease in the number of cells in the G0/G1 fraction. The general trend in the distribution of cells within the cell cycle indicates that timing of the cell cycle is different for chosen cells. Overall analysis of CAP effect on carcinoma, papilloma and glioblastoma cells suggested that the CAP treatment causes significant arrest in the G2/M phase. As such, the present inventors have found that CAP treatment can be combined with chemotherapy and radiation therapy targeting cancer cells in specific phase of the cell cycle.

For example, GBM is a highly malignant very aggressive neoplasm of the central nervous system characterized by rapid growth, extensive angiogenesis, and resistance to all current therapies. Thereby, GBM is associated with poor survival and high mortality where approximately 7,500 patients die from GBM each year. Treatments of GBM tumors remain largely palliative despite recent advances with the integration of multi-modal therapies. The standard of care for newly diagnosed GBM is maximal surgical resection followed by six cycles of concurrent radiation and chemotherapy (i.e., Temozolamide, TMZ), which has been shown to only improve median survival up to 14.6 months and a continued dismal five-year survival at less than 10%. The major limitations of tumor treatment and eventual tumor recurrence are: (a) the tumor cells are very resistant to conventional therapies; (b) the brain is susceptible to damage with conventional therapies; (c) the brain has a very limited capacity to repair itself, and (d) many drugs cannot cross the blood-brain barrier to act on the brain tumor. Thus, it is necessary to develop novel tools that can provide selective targeting of proliferating tumor cells and also enhance existing therapies.

To evaluate effect of CAP on brain tumor, a micro-CAP device was developed and directly applied to glioblastoma tumors in the brain of living mice via an implanted endoscopic tube. See U.S. Published Patent Application No. 2018/0271579. Using in vivo bioluminescence imaging, the tumor volume in a control animal (helium only) increased nearly 600% over the course of two days, whereas micro-CAP-treated tumor volume decreased approximately 50% compared with baseline levels. In the control animals, there is a clear increase in tumor volume over the 2-day period. In contrast, micro-CAP-treated animals fell below baseline values. These striking findings demonstrate the potential of CAP to inhibit glioblastoma tumor growth in vivo. Given these findings, it can be suggested that cold plasma represents a promising new adjunct for cancer therapy, offering the ability to directly target and selectively kill neoplastic tissue. Notably, CAP device provides a method for practical administration of this cancer therapy. Plasma therapy could potentially target internal malignancies via an endoscopic delivery system, thus enabling this technology to serve as either a standalone treatment option or, more realistically, an adjuvant to existing therapies.

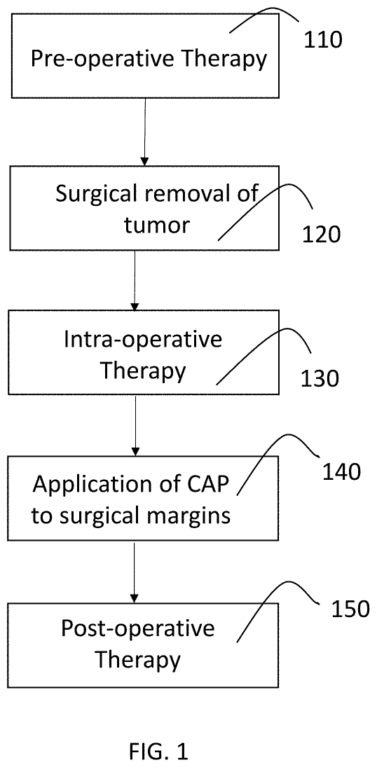

A method for treating solid tumors with a combination of CAP and other therapies in accordance with a preferred embodiment of the present invention is shown in FIG. 1. A patient having a solid cancerous tumor is treated with at least one of systemic chemotherapy or external beam radiation therapy. The systemic chemotherapy or external beam radiation therapy is started on the patient pre-operatively (110). The cancerous tumor is then surgically removed from the patient (120). Surgical removal may be performed by any means, including open surgery, laparoscopic surgery, endoscopic surgery. The systemic chemotherapy or beam radiation therapy may be continued intra-operatively (130). Cytoreductive surgery (CRS), and/or hyperthermic intraperitoneal chemotherapy (HIPEC) also may be performed. Cold atmospheric plasma is applied to the surgical margins surrounding the area in the patient from which the tumor was removed (140). The systemic chemotherapy or external beam radiation therapy is then continued post-operatively (150).

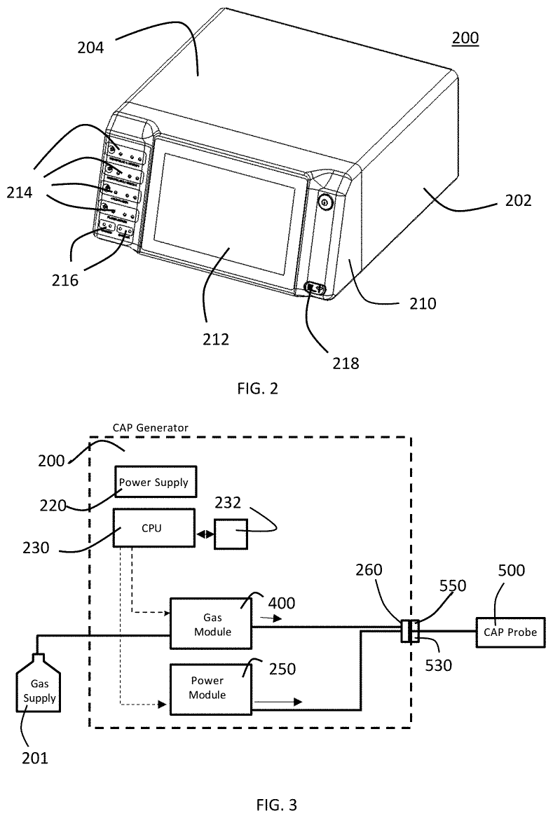

A preferred embodiment of a CAP enabled generator is described with reference to the drawings. A gas-enhanced electrosurgical generator 200 in accordance with a preferred embodiment of the present invention is shown in FIGS. 2 and 3. The gas-enhanced generator has a housing 202 made of a sturdy material such as plastic or metal similar to materials used for housings of conventional electrosurgical generators. The housing 202 has a removable cover 204. The housing 202 and cover 204 have means, such as screws, tongue and groove, or other structure for removably securing the cover to the housing. The cover 204 may comprise just the top of the housing or multiple sides, such as the top, right side and left side, of the housing 202. The housing 202 may have a plurality of feet or legs (not shown) attached to the bottom of the housing. The bottom of the housing 202 may have a plurality of vents (not shown) for venting from the interior of the gas-enhanced generator.

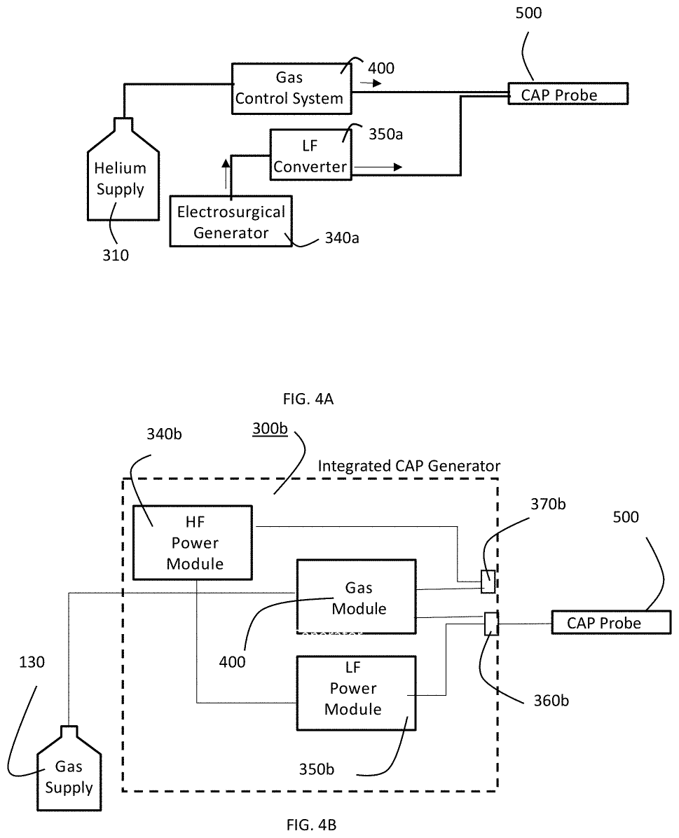

A generator housing front panel 210 is connected to the housing 202. On the face front panel 210 there is a touchscreen display 212 and there may be one or a plurality of connectors 214 for connecting various accessories to the generator 200. For a cold atmospheric plasma generator such as is shown in FIG. 3, for example, there is a connector 260 for connecting a cold atmospheric probe 500. An integrated multi-function electrosurgical generator, such as is shown in FIG. 4B the plurality of connectors may include an argon plasma probe, a hybrid plasma probe, a cold atmospheric plasma probe, or any other electrosurgical attachment. The face of the front panel 210 is at an angle other than 90 degrees with respect to the top and bottom of the housing to provide for easier viewing and use of the touch screen display 212 by a user.

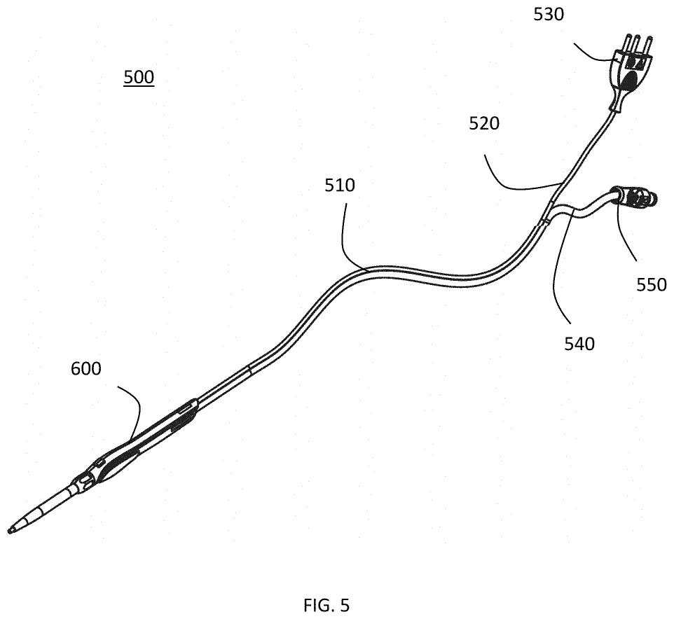

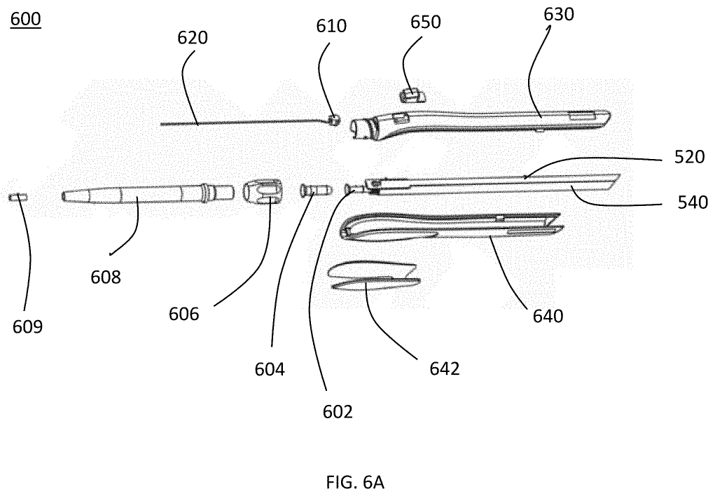

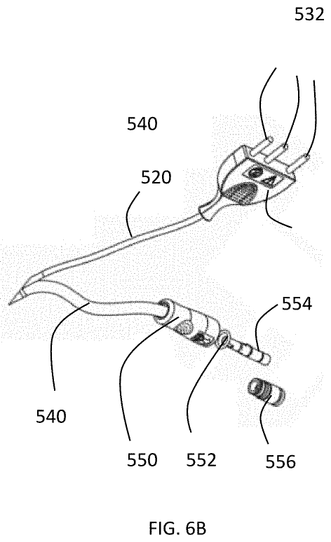

As shown in FIG. 3, an exemplary cold atmospheric plasma (CAP) generator 200 has a power supply 220, a CPU (or processor or FPGA) 230 and a memory or storage 232. The system further has a display 212 (FIG. 2), which may be the display of a tablet computer. The CPU 230 controls the system and receives input from a user through a graphical user interface displayed on display 212. The CAP generator further has a gas control module 400 connected to a source 201 of a CAP carrier gas such as helium. The gas control module 400 may be, for example, of the design described in International Patent Application No. WO 2018/191265, which is hereby incorporated by reference. The CAP generator 200 further has a power module 250 for generating low frequency radio frequency (RF) energy, such as is described in U.S. Pat. No. 9,999,462, which is hereby incorporated by reference in its entirety. The power module 250 contains conventional electronics and/or transformers such as are known to provide RF power in electrosurgical generators. The power module 250 operates with a frequency between 10-200 kHz, which is referred to herein as a “low frequency,” and output peak voltage from 3 kV to 6 kV and preferably at a frequency near (within 20%) of 40 Hz, 100 Hz or 200 Hz. The gas module 400 and power module 250 are connected to connector 260 that allows for attachment of a CAP applicator 500 (as shown in FIGS. 5, 6A and 6B) to be connected to the generator 200 via a connector having an electrical connector 530 and gas connector 550.

As shown in FIG. 4B, other arrangements for delivery of the carrier gas and the electrical energy may be used with the invention. In FIG. 4B, an integrated CAP generator 300b is connected to a source 310 of a carrier gas (helium in this example), which is provided to a gas control system 400, which supplies the gas at a controlled flow rate to CAP applicator 500. A high frequency (HF) power module 340b supplies high frequency (HF) energy to a low frequency power module (converter) 350b, which outputs electrical energy having a frequency in the range of 10 kHz to 200 kHz and an output voltage in the range of 3 kV to 6 Kv. This type of integrated generator will have both a CAP connector 360b for connecting a CAP applicator or other CAP accessory and a connector 370b for attaching HF electrosurgical attachments such as an argon plasma or hybrid plasma probe (not shown).

Another embodiment, shown in FIG. 4A, has a carrier gas source 310 connected to a conventional gas control system 370, which in turn is connected to the CAP applicator 500, and a conventional electrosurgical generator 340 connected to a low frequency (LF) converter 350a, which is then connected to the CAP probe 500.

In the above-disclosed embodiment, a cold atmospheric plasma below 35° C. is produced. When applied to the tissue surrounding the surgical area, the cold atmospheric plasma induces metabolic suppression in only the tumor cells and enhances the response to the drugs that are injected into the patient.

The cold plasma applicator 500 may be in a form such as is disclosed in U.S. Pat. No. 10,405,913 and shown in FIGS. 5, 6A and 6B. A hand piece assembly 600 has a top side piece 630 and a bottom side piece 640. A control button 650 extends from the interior of the hand piece through an opening in the top side piece 630. Within the hand piece 600 is body connector funnel 602, PCB board 608, electrical wiring 520 and hose tubing (PVC medical grade) 540. The wiring 520 and hose tubing 540 are connected to one another to forma wire and tubing bundle 510. A grip over mold 642 extends over the bottom piece portion 640. In other embodiments, a grip may be attached to the bottom piece 640 in other manners. A probe or scalpel assembly is attached to the end of the hand piece. The probe assembly has non-bendable telescoping tubing 606, a ceramic tip 609, a column nut or collet 606 and body connector tubing 604. The hose tubing 540 extends out of the proximal end of the hand piece to a body gas connector 550, which has an O-ring 552, gas connector core 554 and gas connector tip 556 for connecting to a connector on a gas-enhanced electrosurgical generator. The printed circuit board 608 connects to electrical wiring 520 which leads to electrical connector 530 having electrical pins 532. Inside the handpiece 600 is an electrode 620 and conductive connector 610. There is a control button 650 for controlling the application of electrical energy.

### Experiments

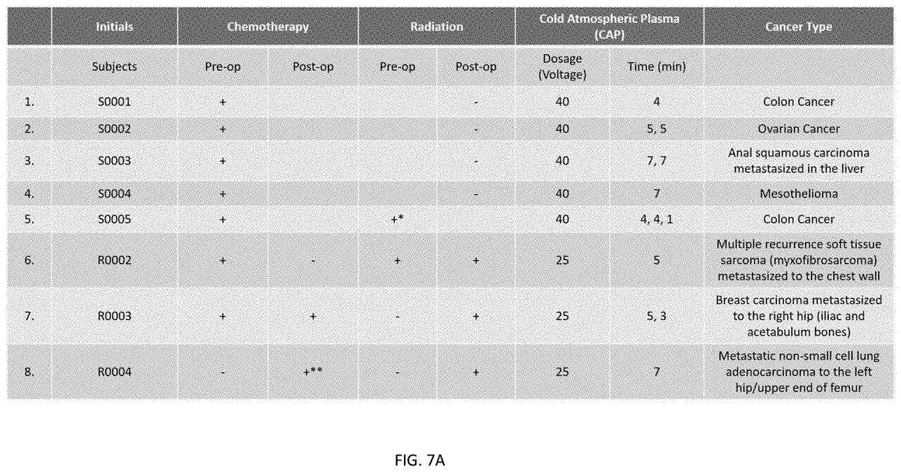

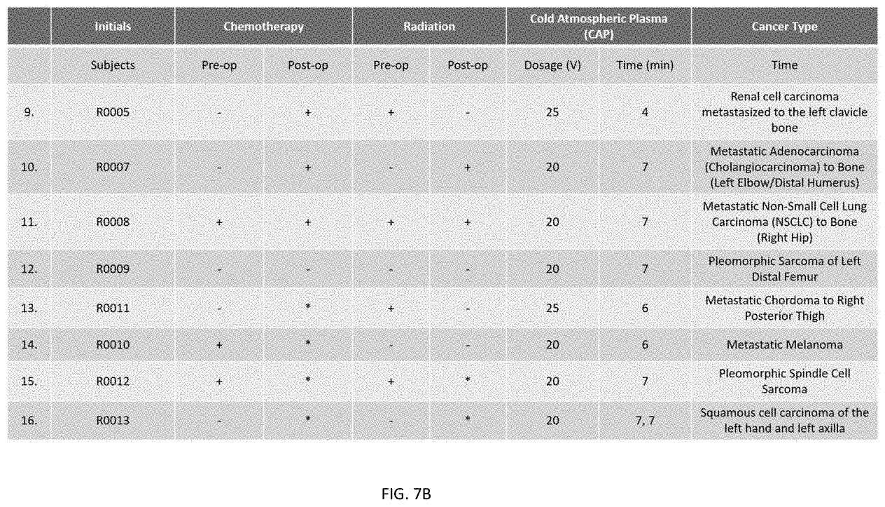

As summarized in FIGS. 7A and 7B, sixteen patients were treated with CAP. As shown in Table 1, the fifteen patients were treated with surgical resection and various combinations of CAP, chemotherapy and radiation therapy and one was treated with surgical resection and CAP only:

| | Pre-op | Pre-op | Intra-op | Intra-op | Intra-op | Post-op | Post-op |
|---|---|---|---|---|---|---|---|
| | Chemo | Radiation | Chemo | Radiation | CAP | Chemo | Radiation |
| 4 | X | | | | X | | |
| 1 | X | | | X | X | | |
| 1 | X | X | | | X | | X |
| 1 | X | | | | X | X | X |
| 3 | | | | | X | X | X |
| 2 | | X | | | X | X | |
| 2 | X | X | | | X | X | X |
| 1 | | | | | X | | |
| 1 | X | | | | | X | |

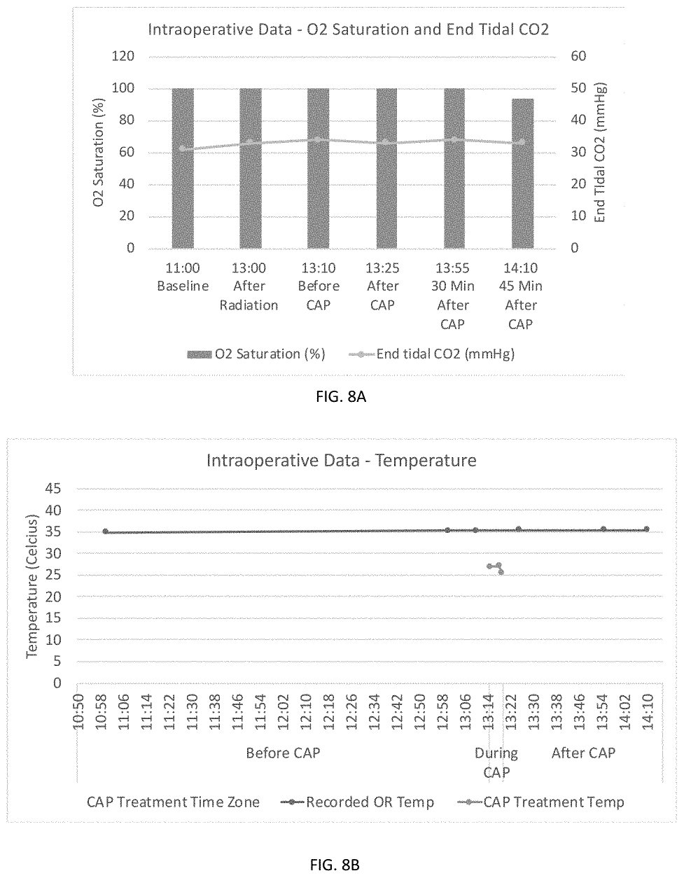

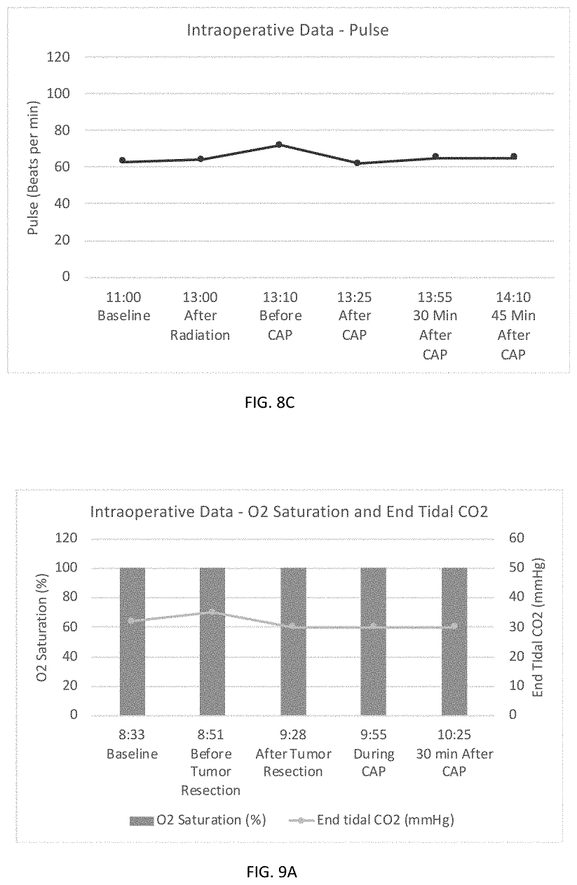

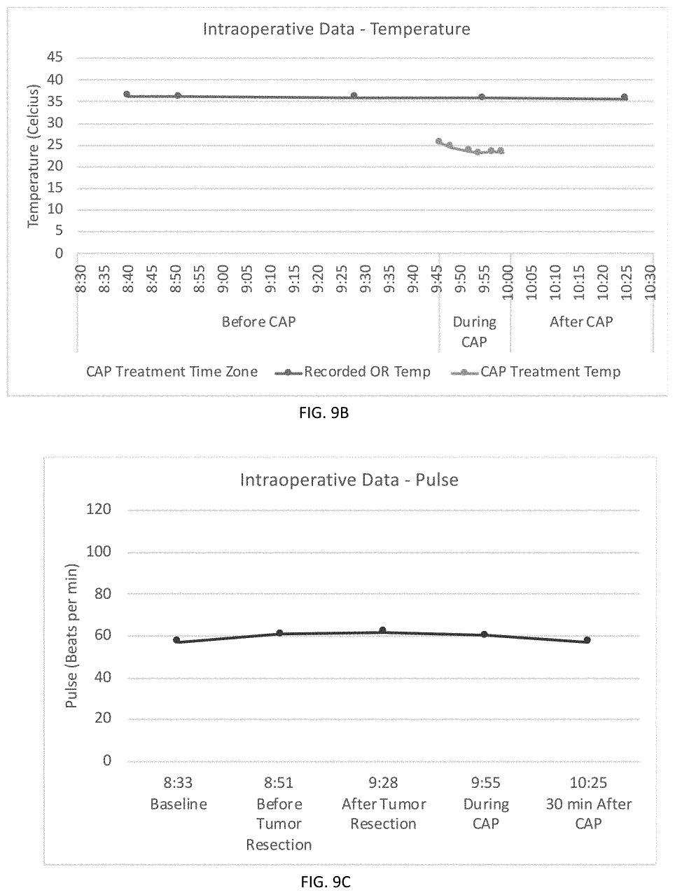

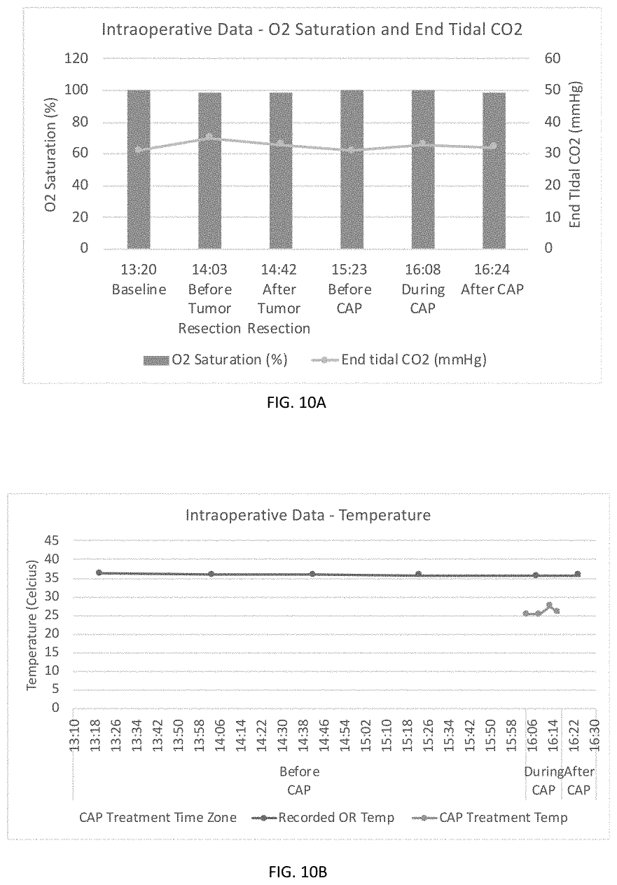

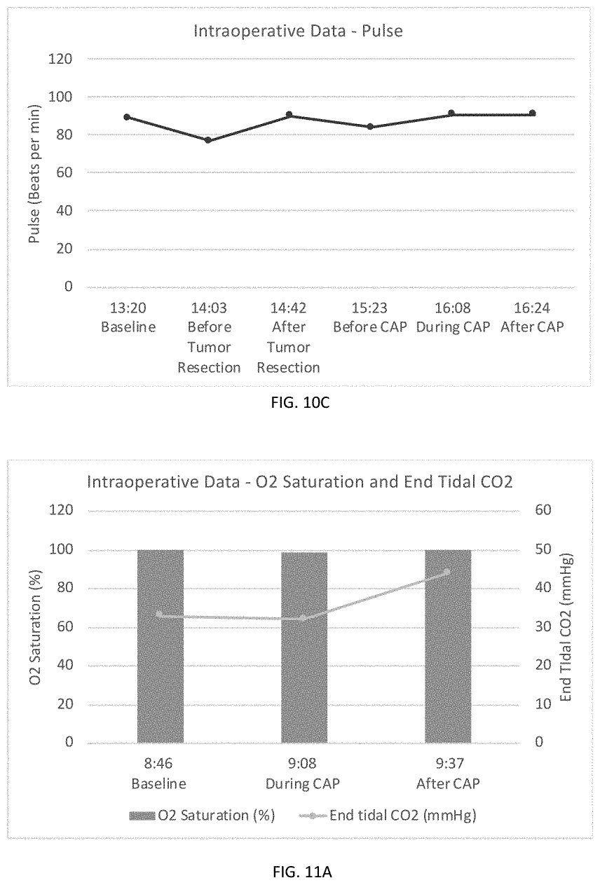

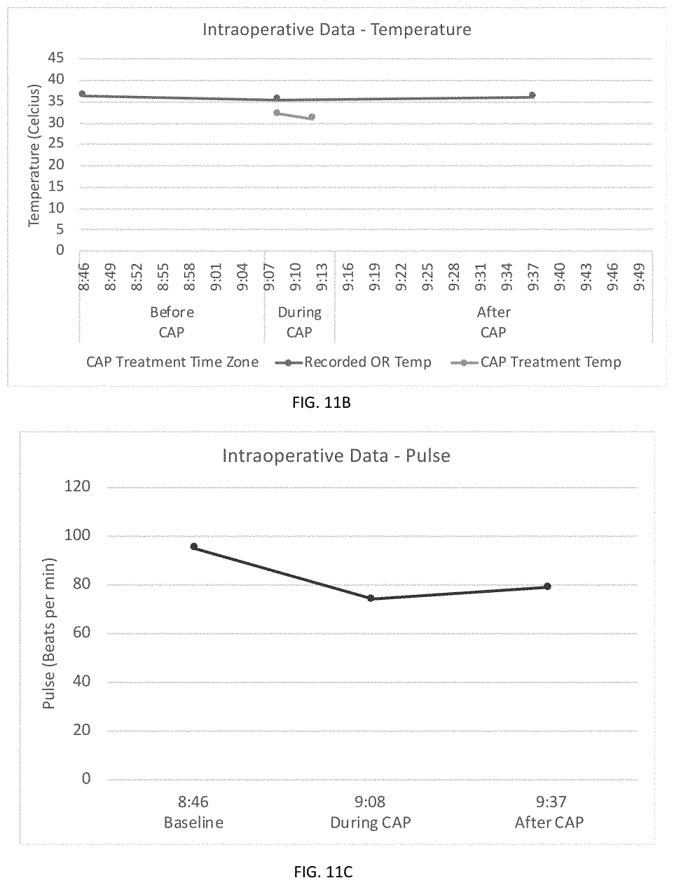

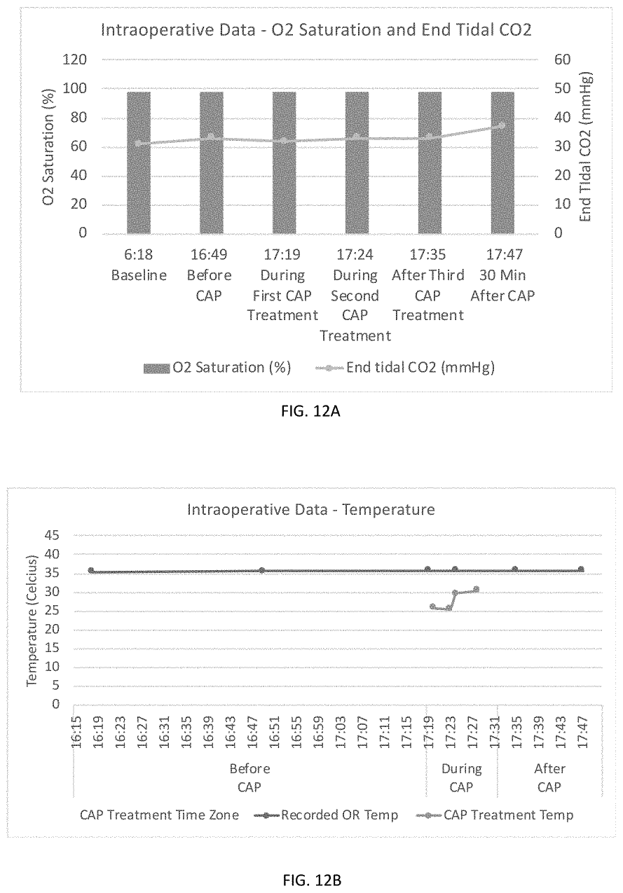

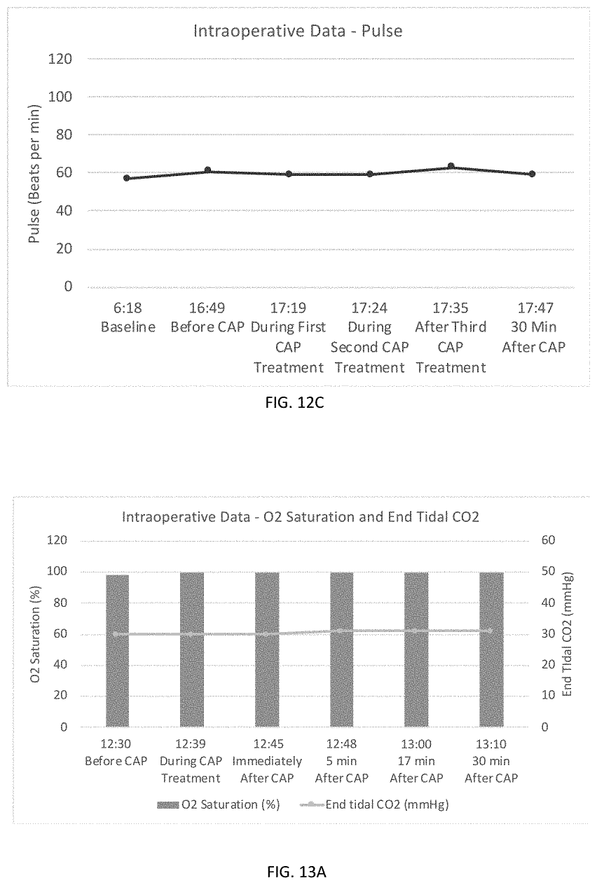

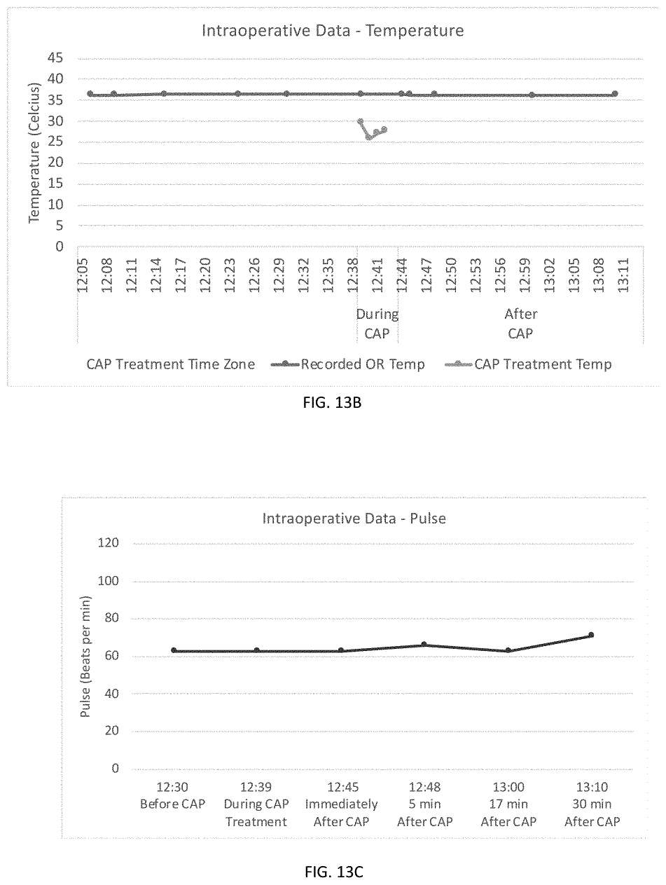

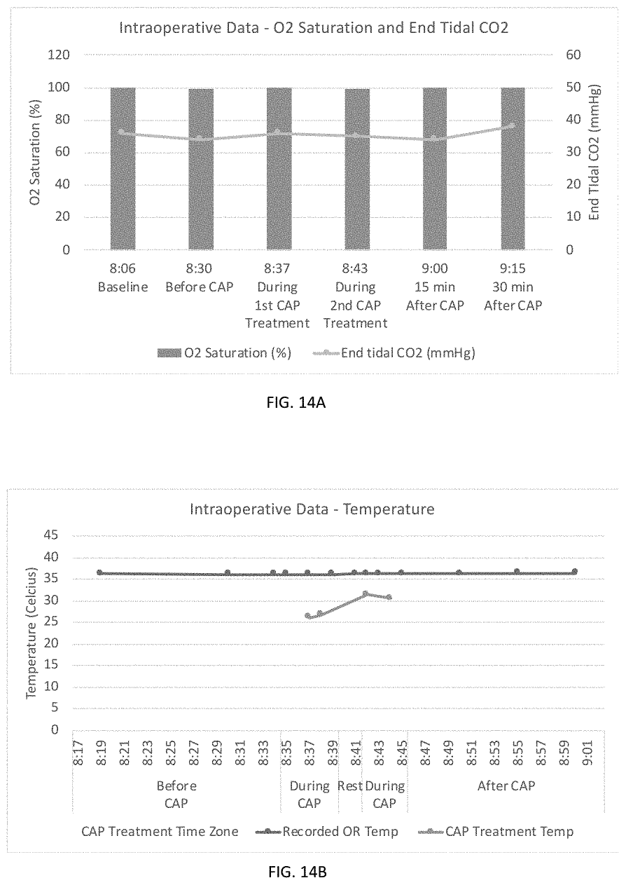

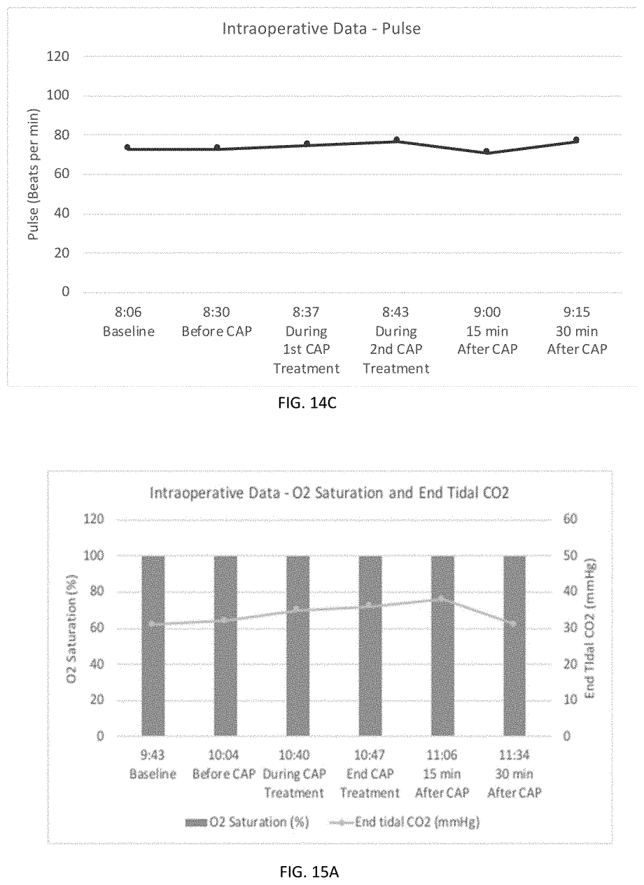

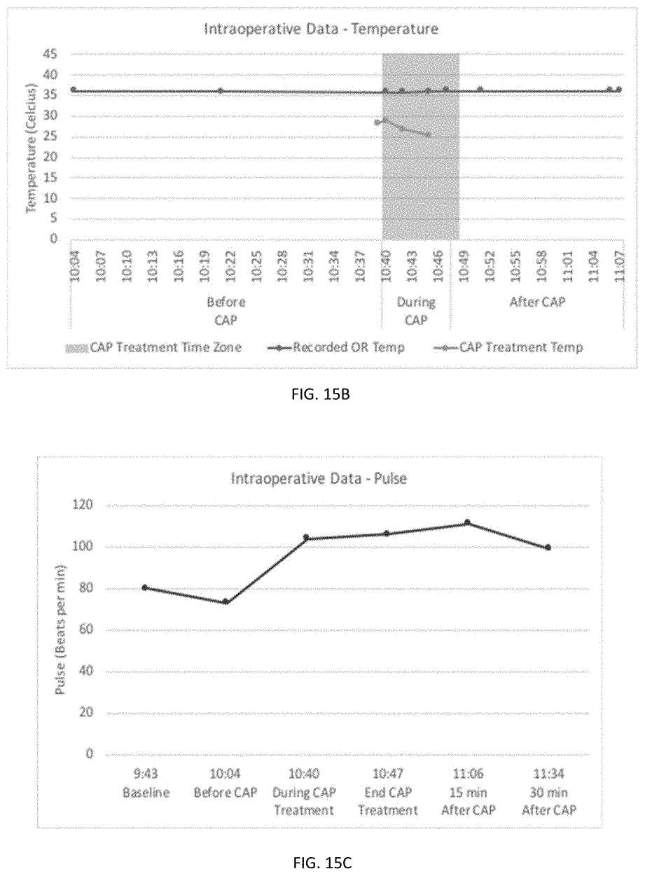

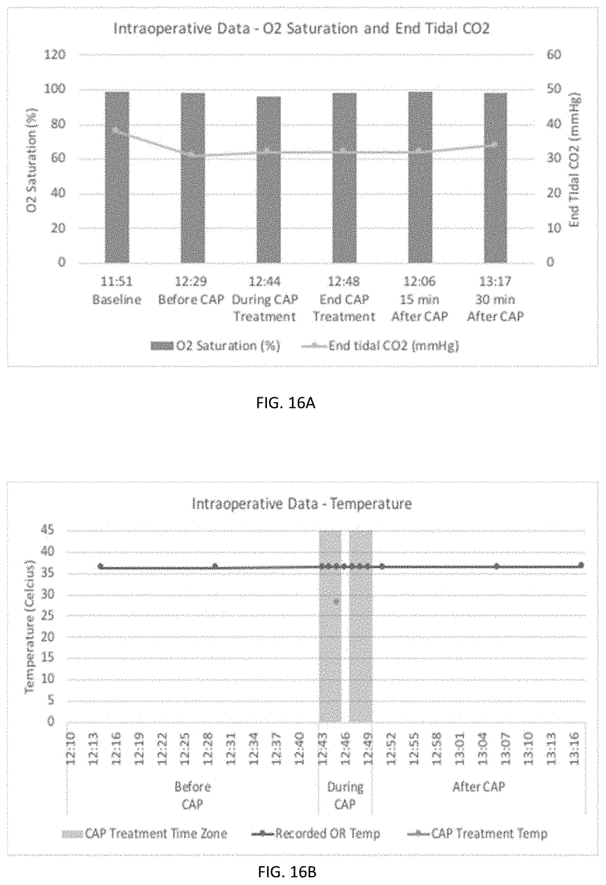

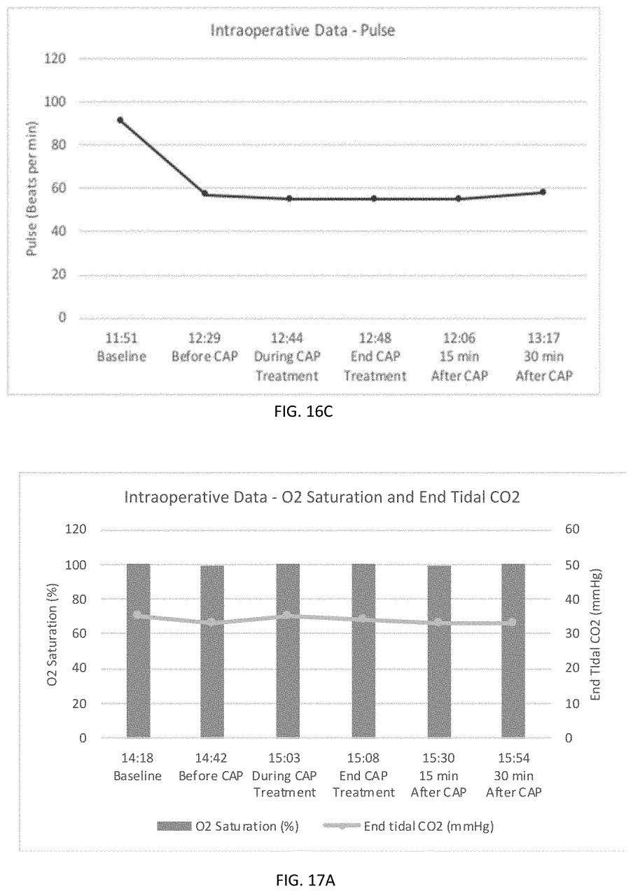

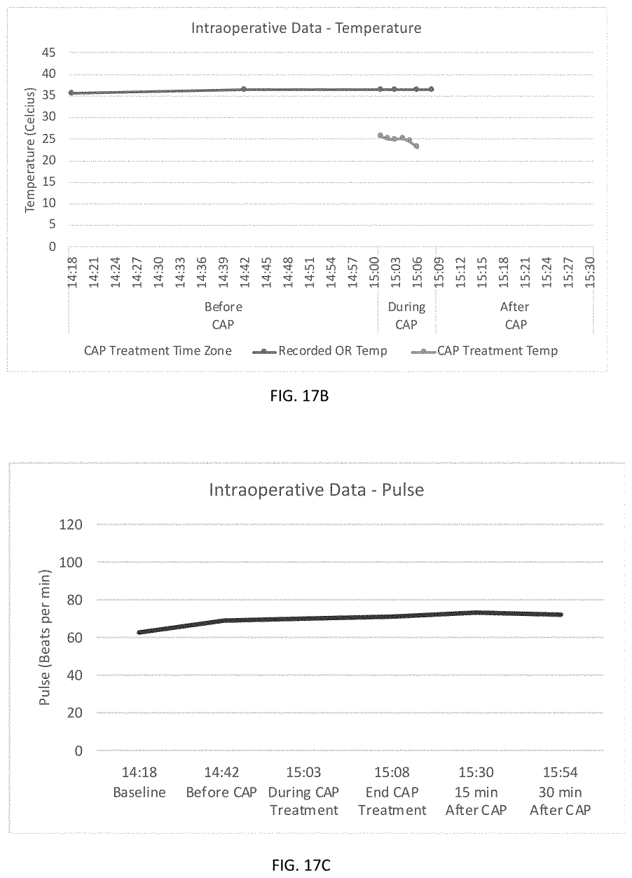

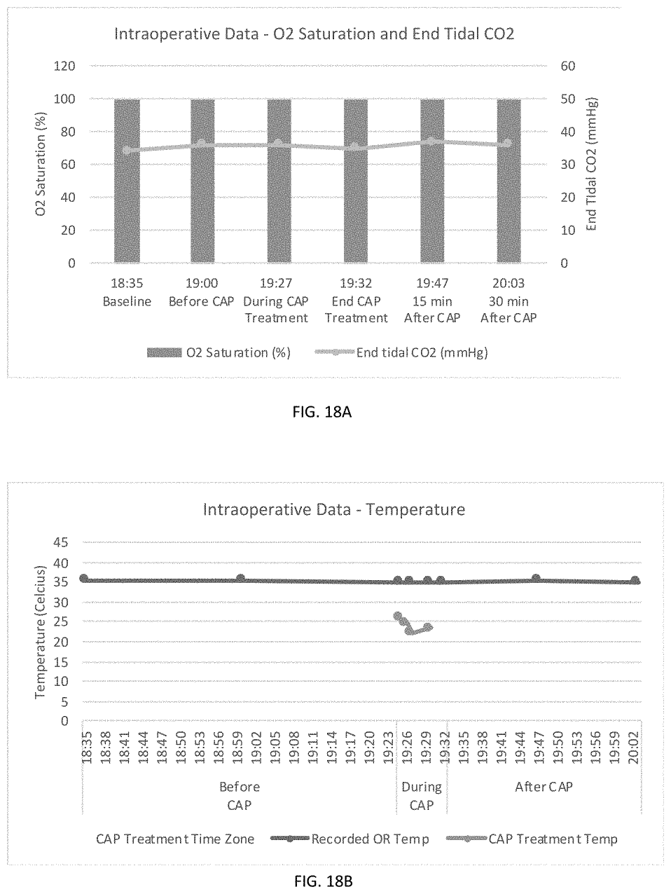

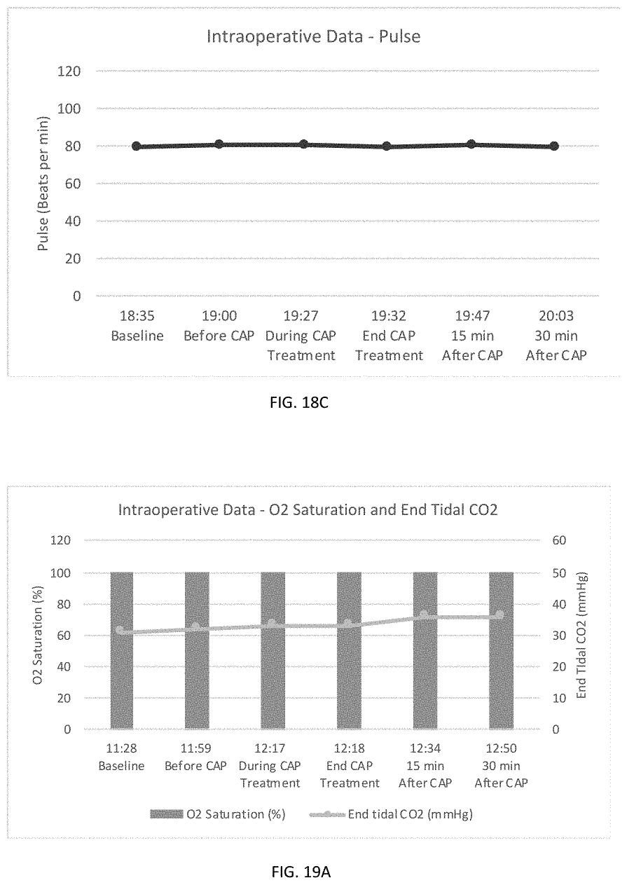

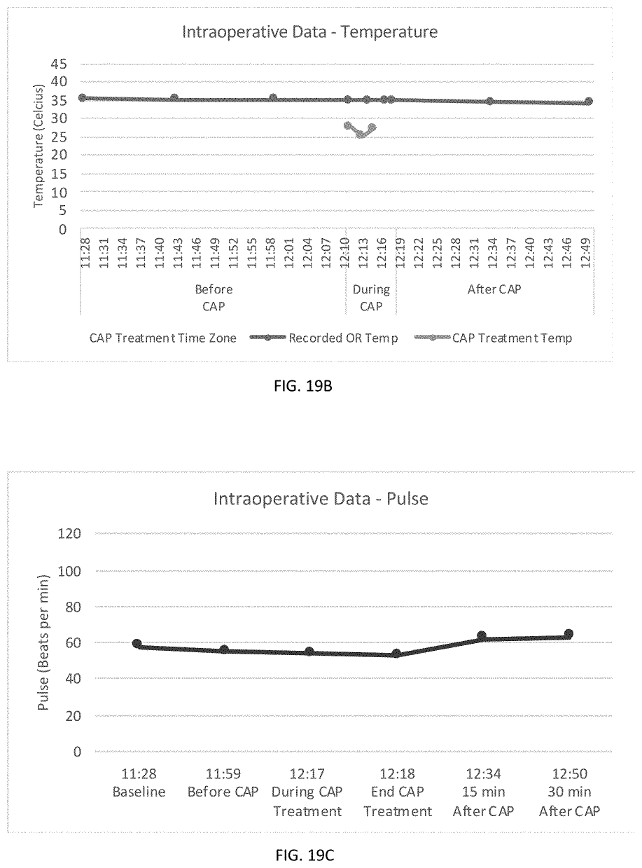

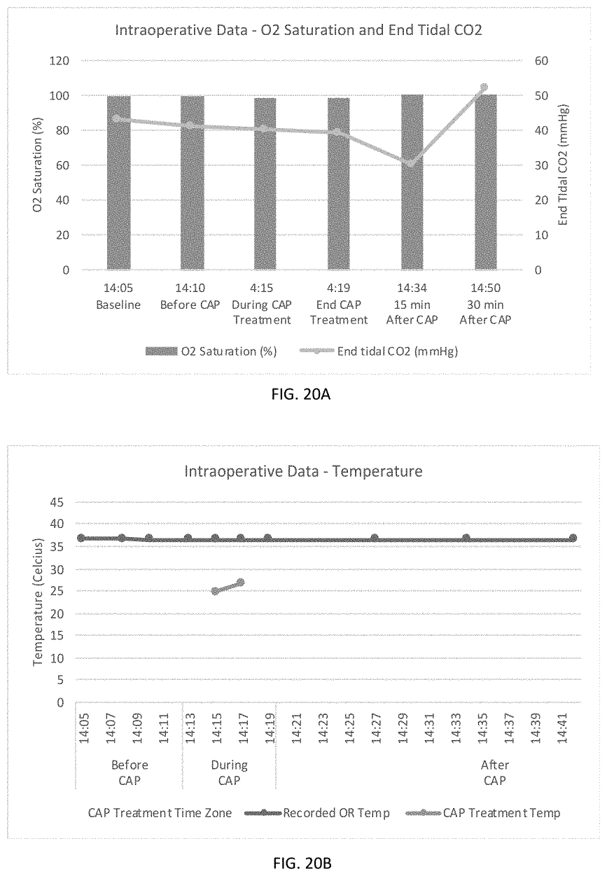

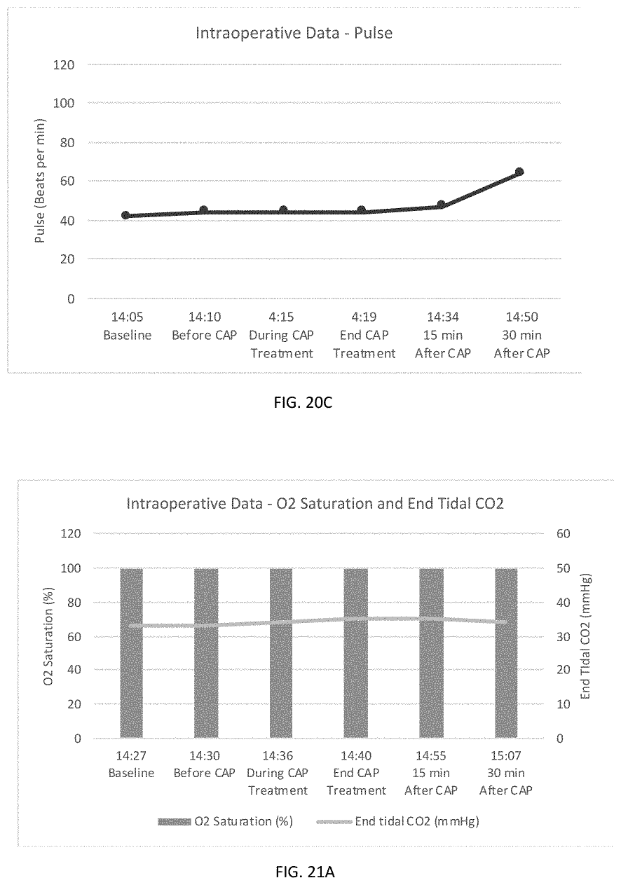

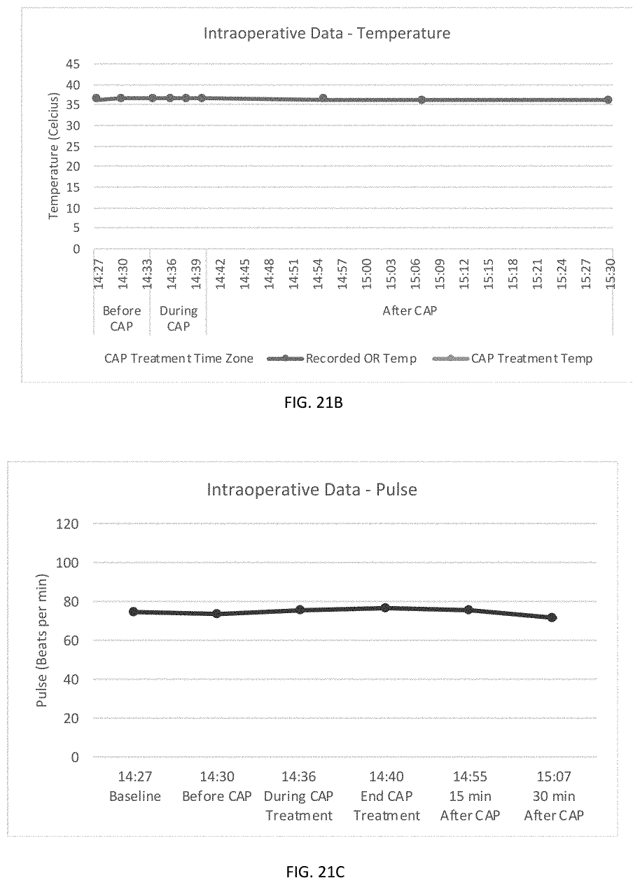

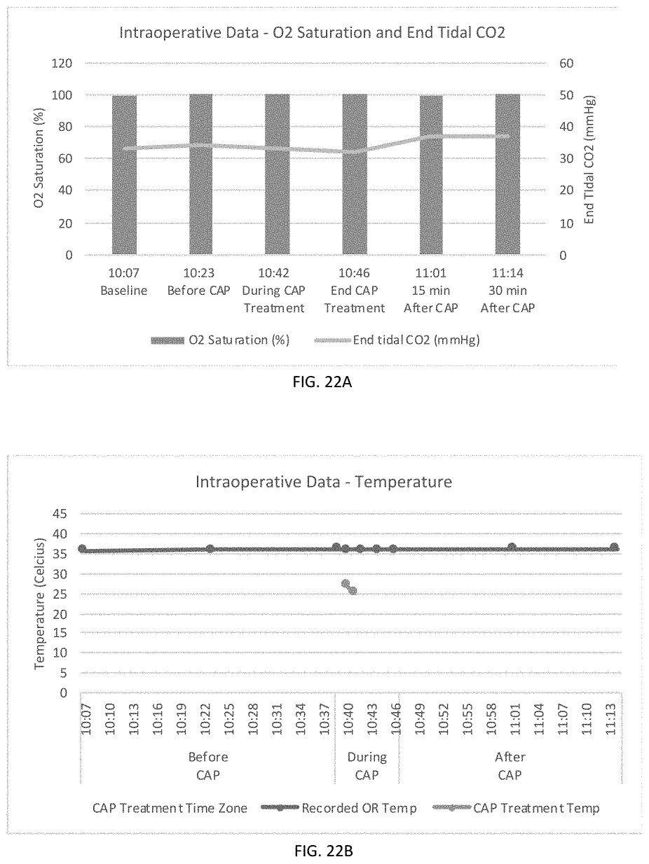

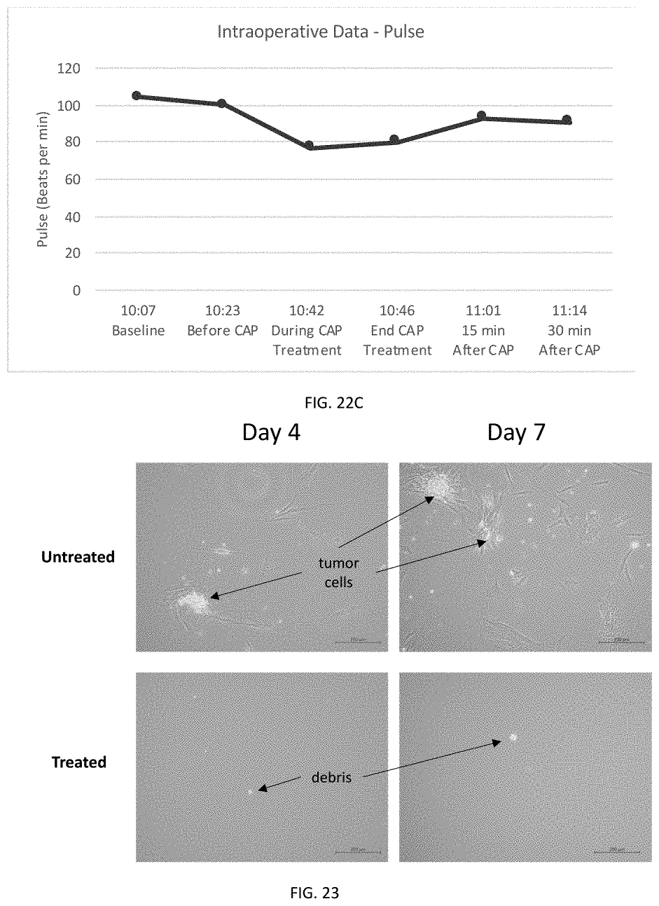

The patients' vital sign statuses were monitored during the surgery and CAP administration. Graphs of the intraoperative data of O2 saturation, body temperature and pulse for the fifteen subjects treated with combination therapy are shown in FIGS. 8A-22C. From the graphs is can be seen that the CAP temperature (25-32 C) is lower than body temperature and the subject vital signs are stable during and after CAP treatment.

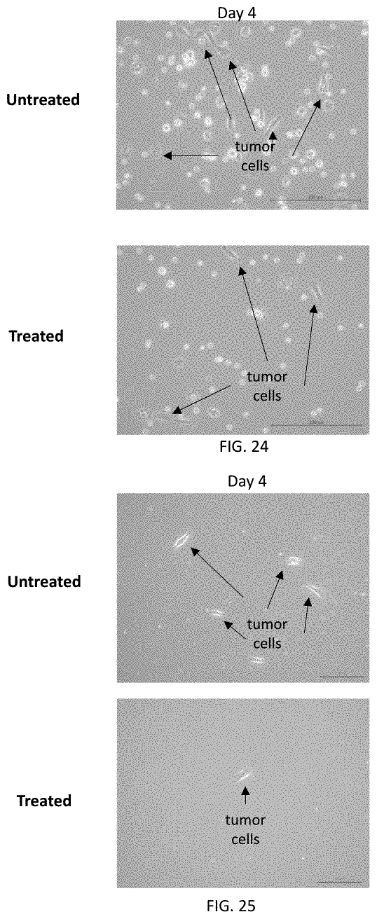

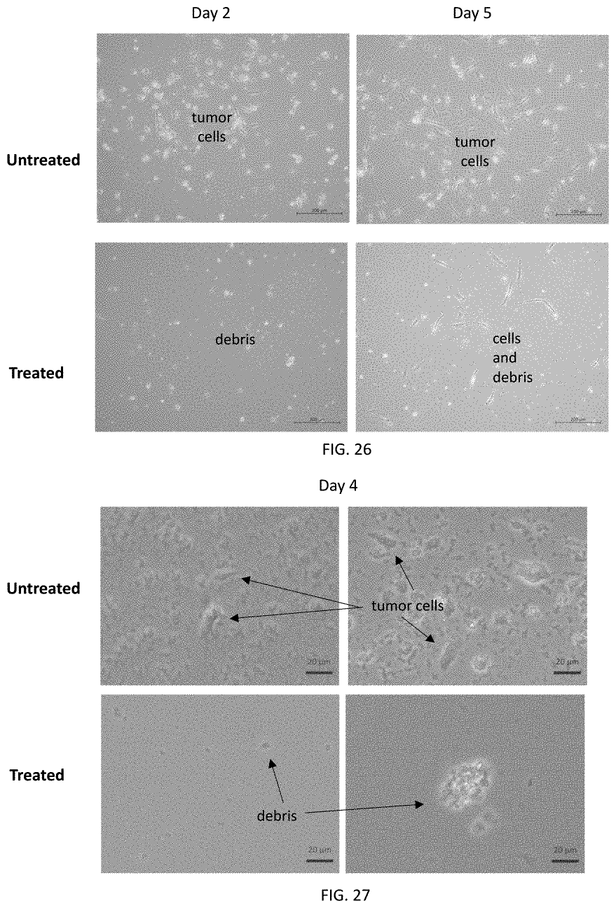

Additionally, subject tumor samples were treated ex vivo with CAP at the time of surgery and dissociated and cultured in the lab. Images of cells were taken for both treated and untreated samples. FIG. 23 is a set of culture images of treated and untreated cancer cells from Subject 6 (R0002), who was treated for myxofibrosarcoma. FIG. 24 is a set of culture images of treated and untreated cancer cells from Subject 10 (R0007), who was treated for cholangiocarcinoma. FIG. 25 is a set of culture images of treated and untreated cancer cells from Subject 12 (R0009), who was treated for pleomorphic sarcoma. FIG. 26 is a set of culture images of treated and untreated cancer cells from Subject 13 (R0011), who was treated for Chordoma. FIG. 27 is a set of culture images of treated and untreated cancer cells from Subject 15 (R0012), who was treated for pleomorphic spindle cell sarcoma.

From the culture images shown in FIGS. 23-27 one can see that for tissue not treated with CAP large heterogenous population of primary cells were adherent and proliferated over time, whereas in the CAP treated tissue few primary cells were adherent and dormient.

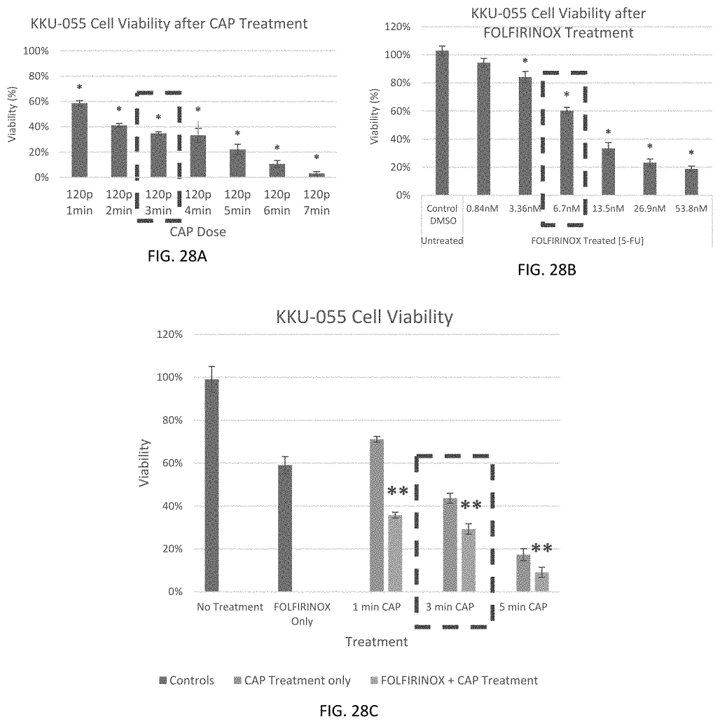

FIGS. 28A-28C are graphs illustrating the synergistic effect between CAP one and FOLFIRINOX. The “**” in FIGS. 28A-28C note statistical significance (p<0.05) where the combination therapy reduces cell viability significantly more than CAP treatment alone and FOLFIRINOX treatment alone.

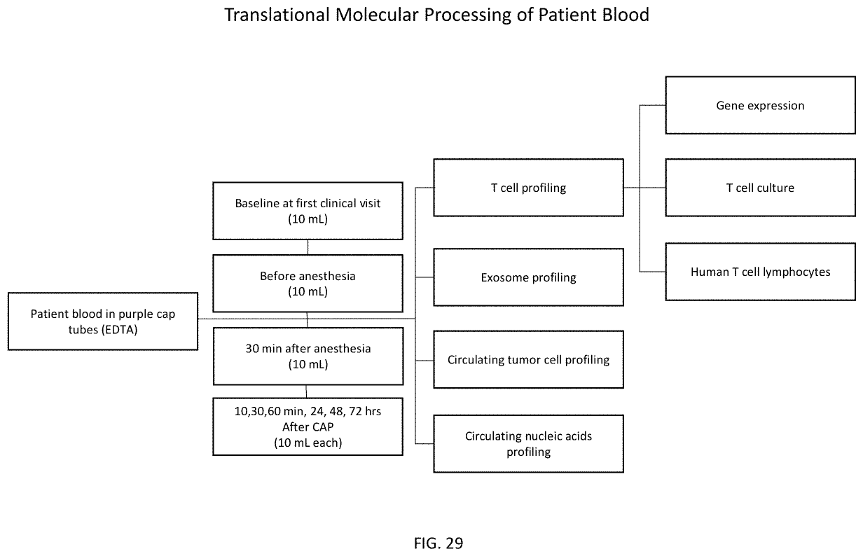

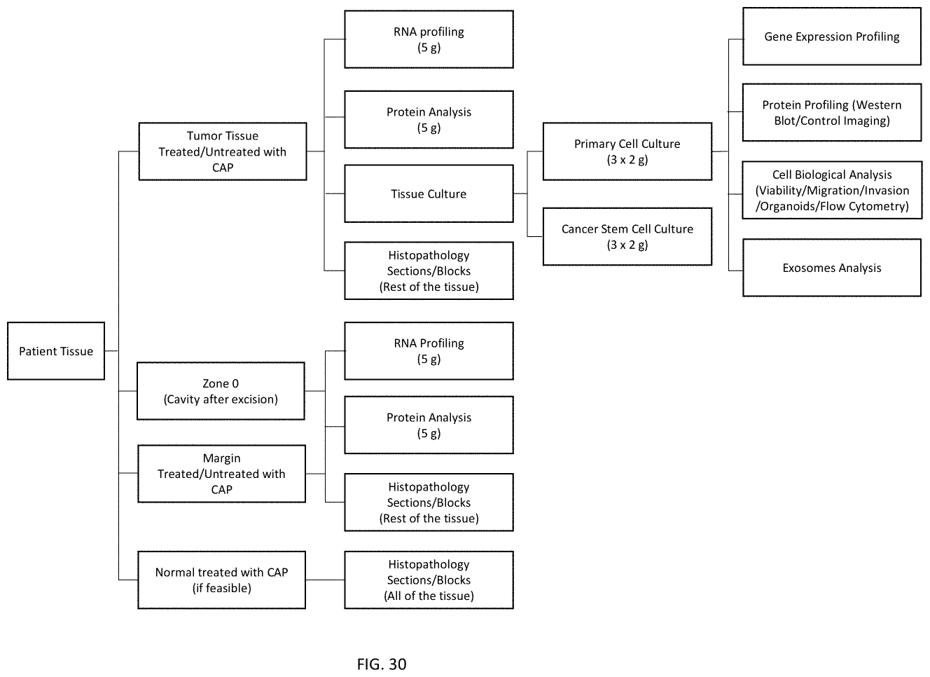

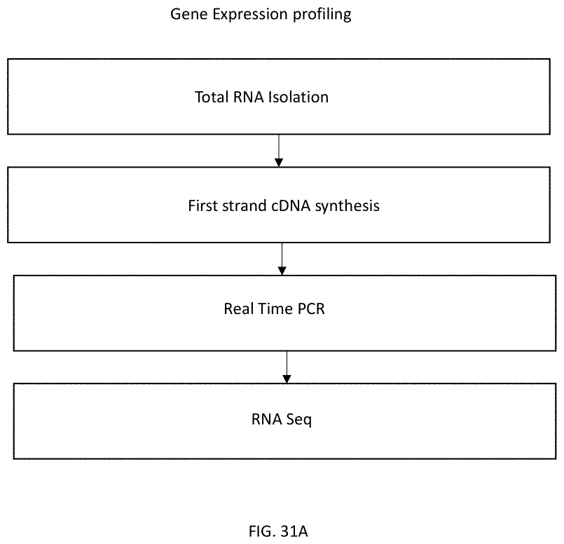

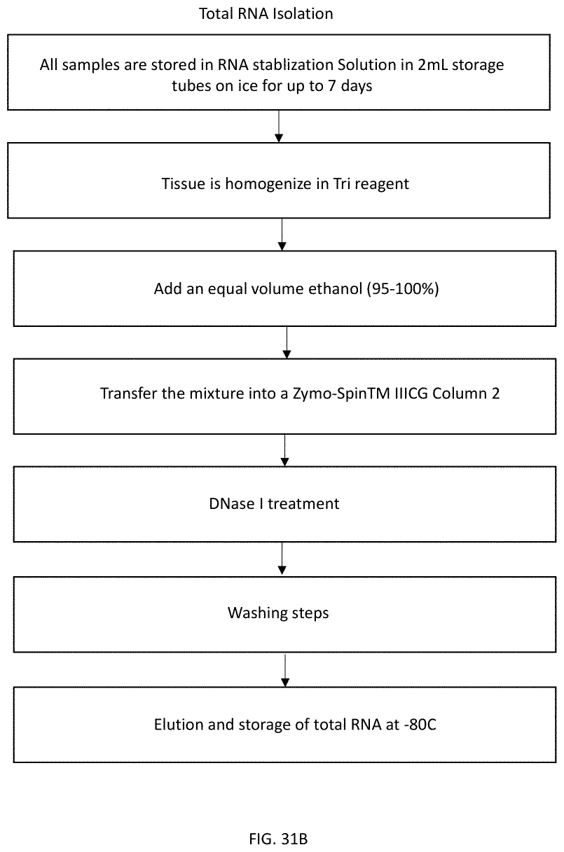

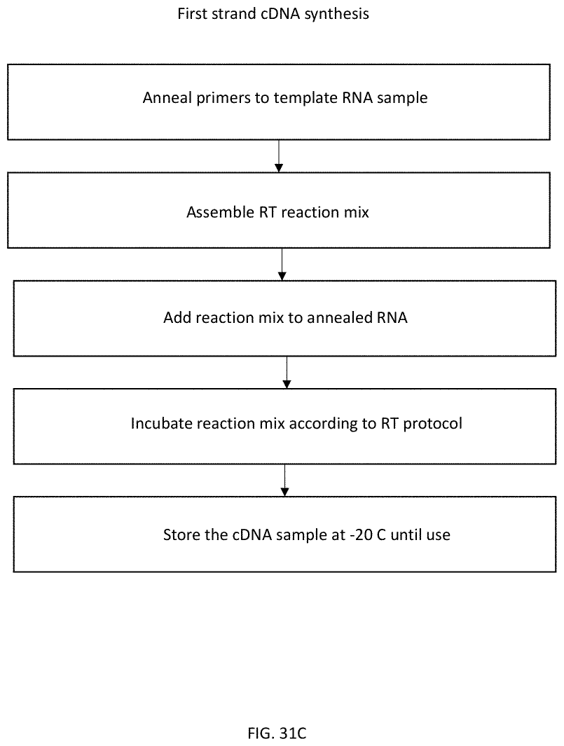

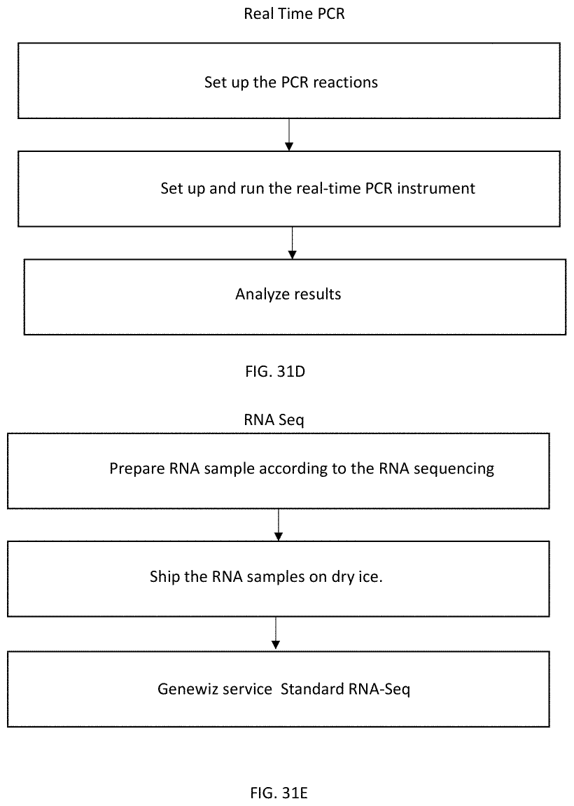

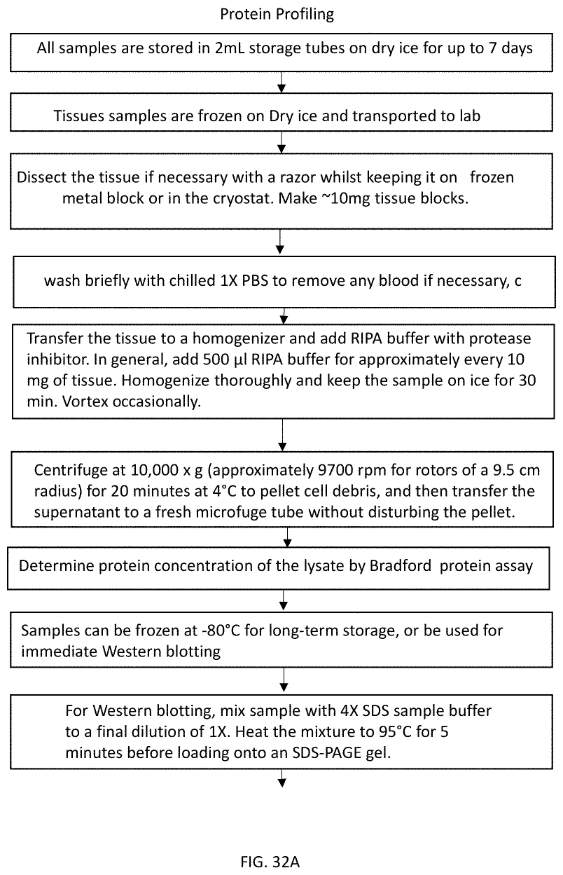

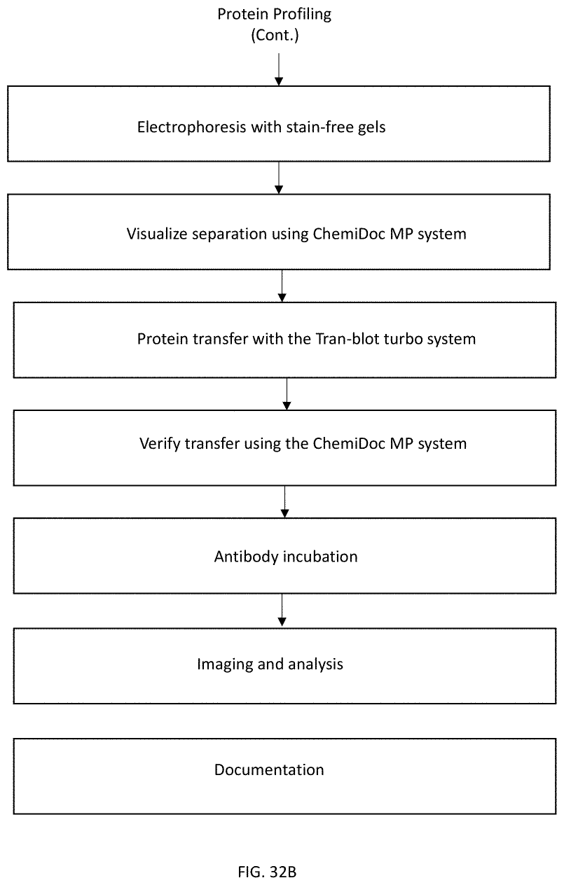

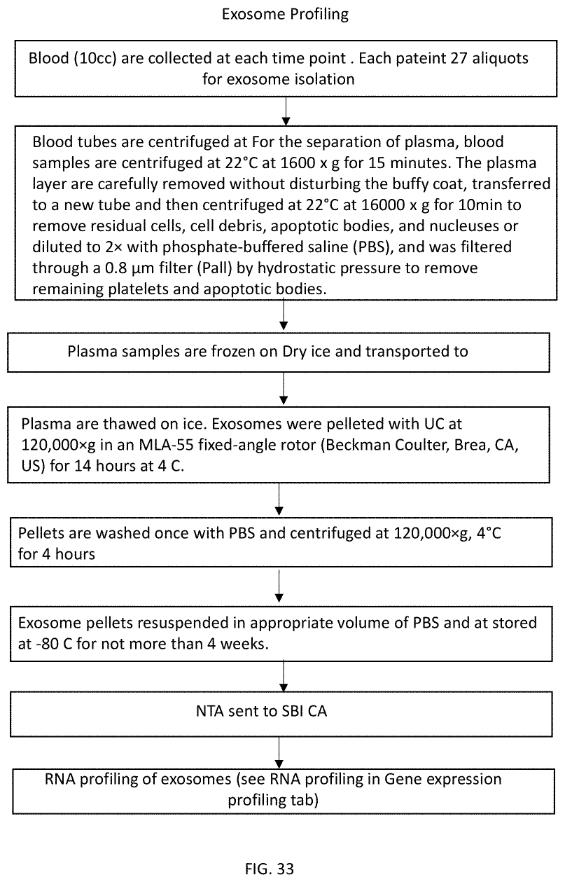

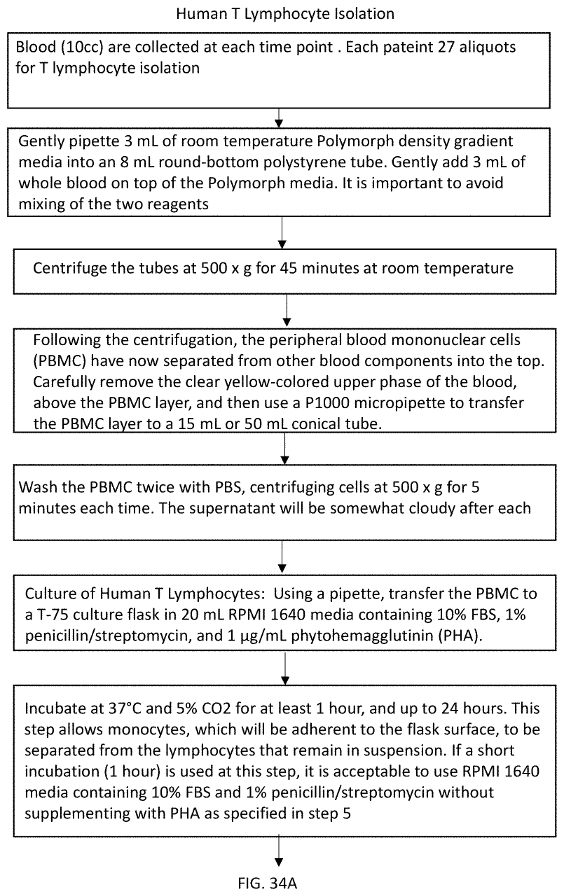

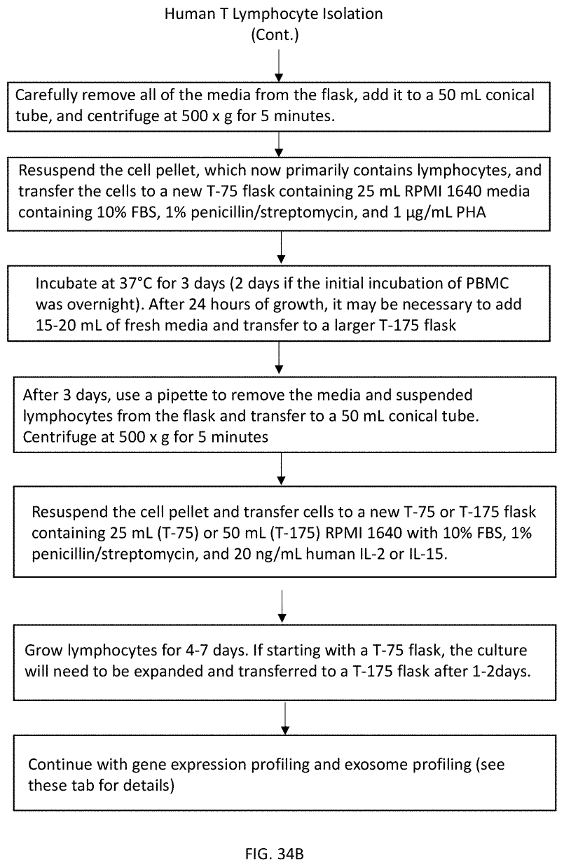

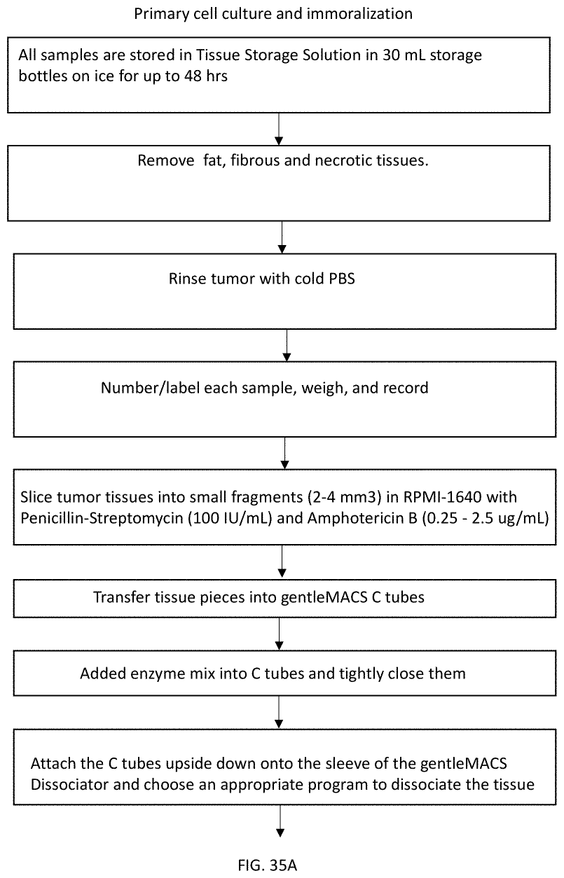

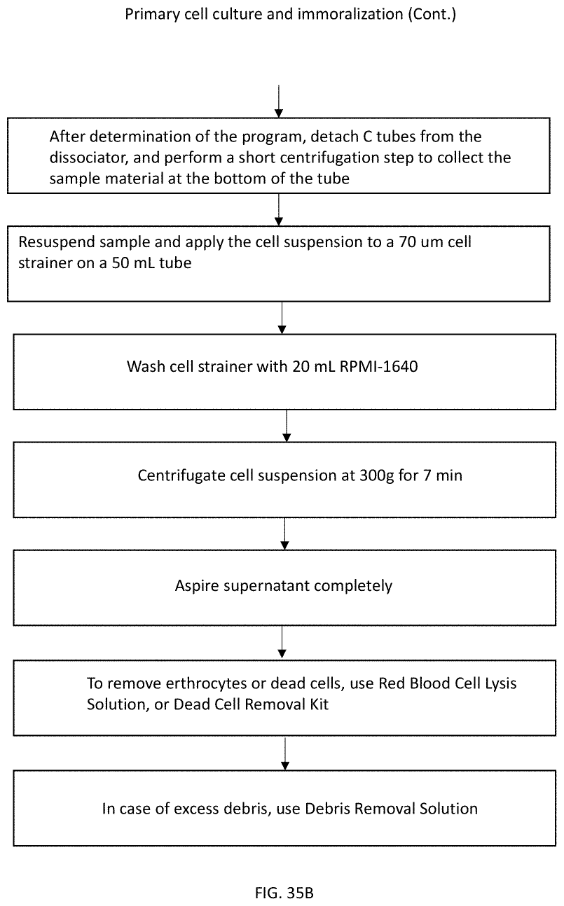

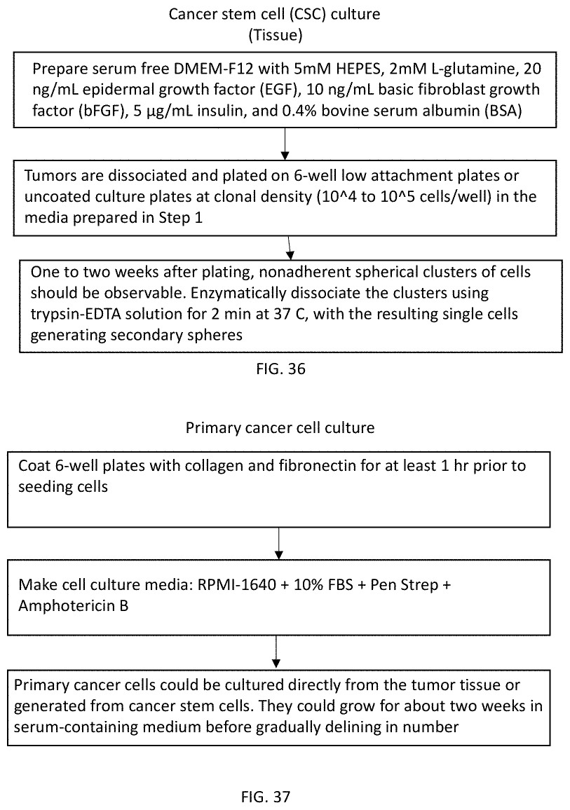

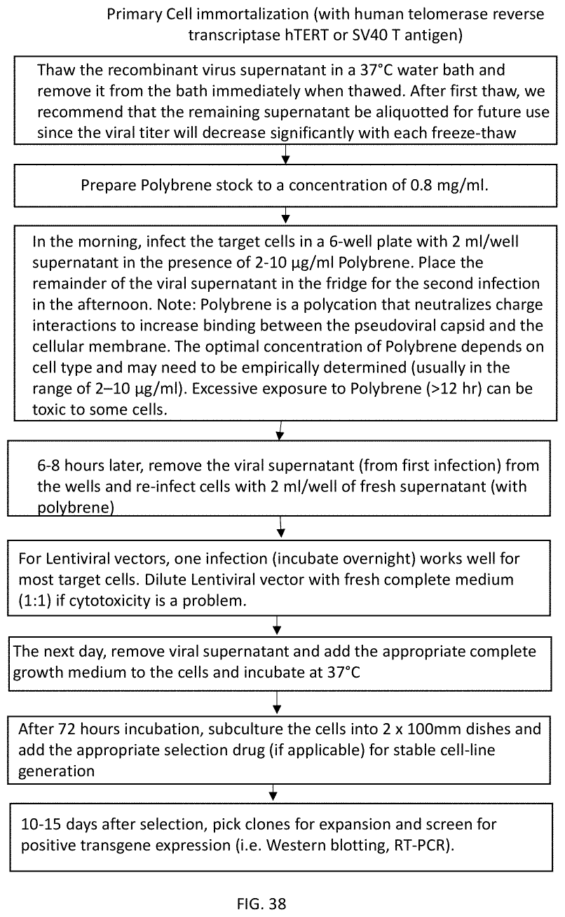

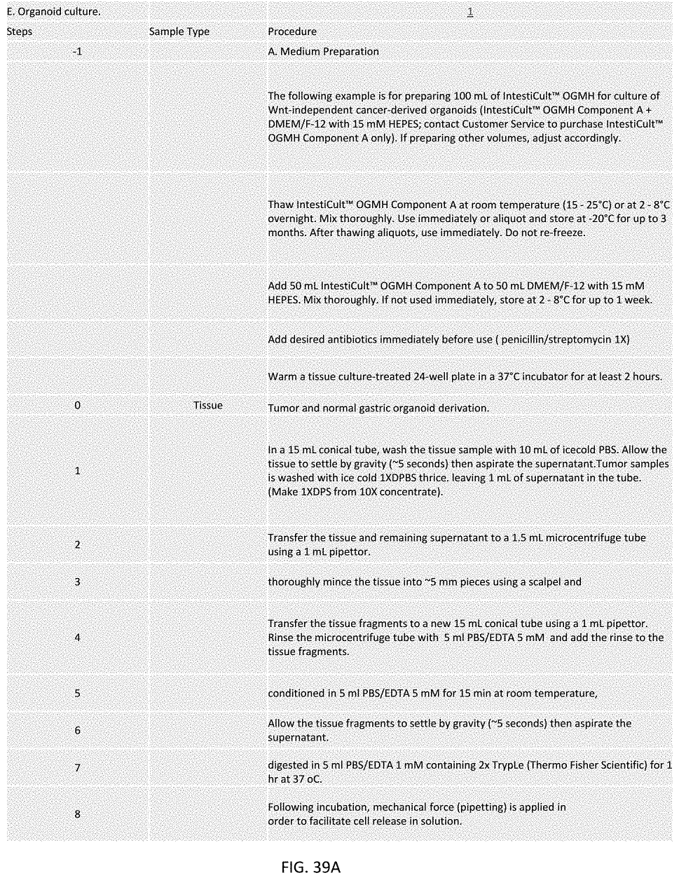

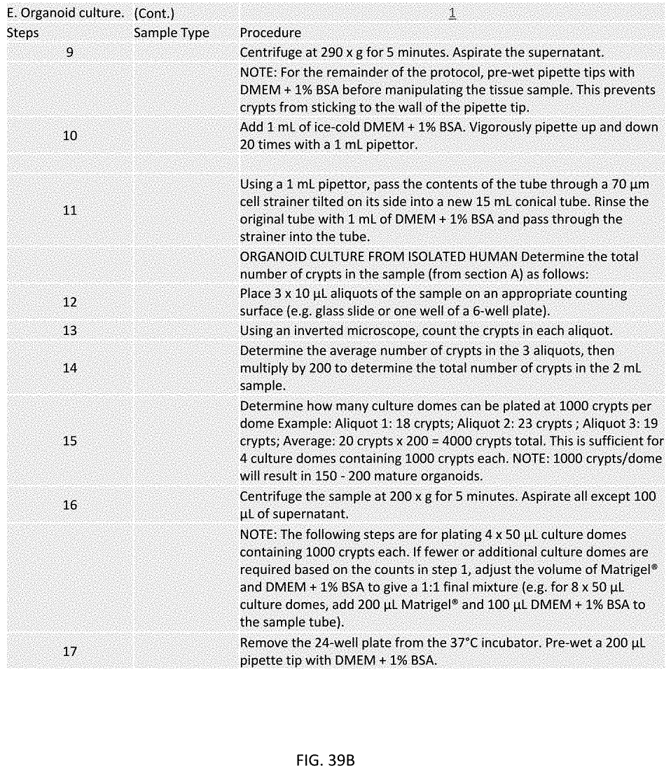

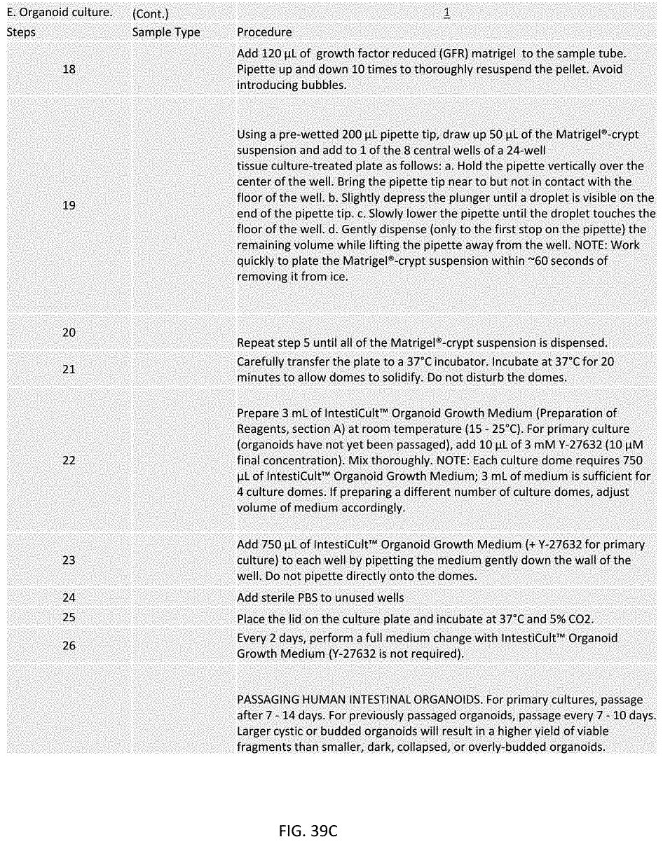

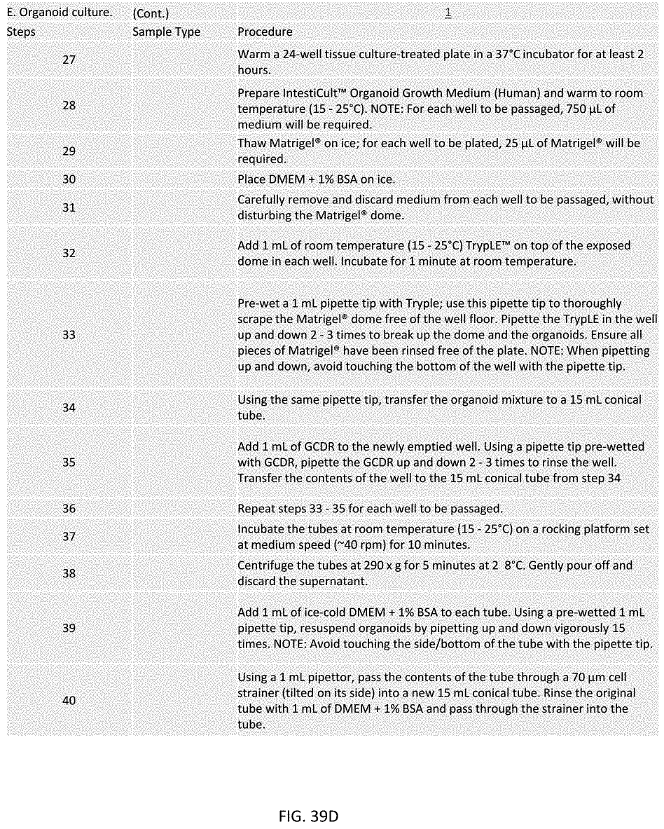

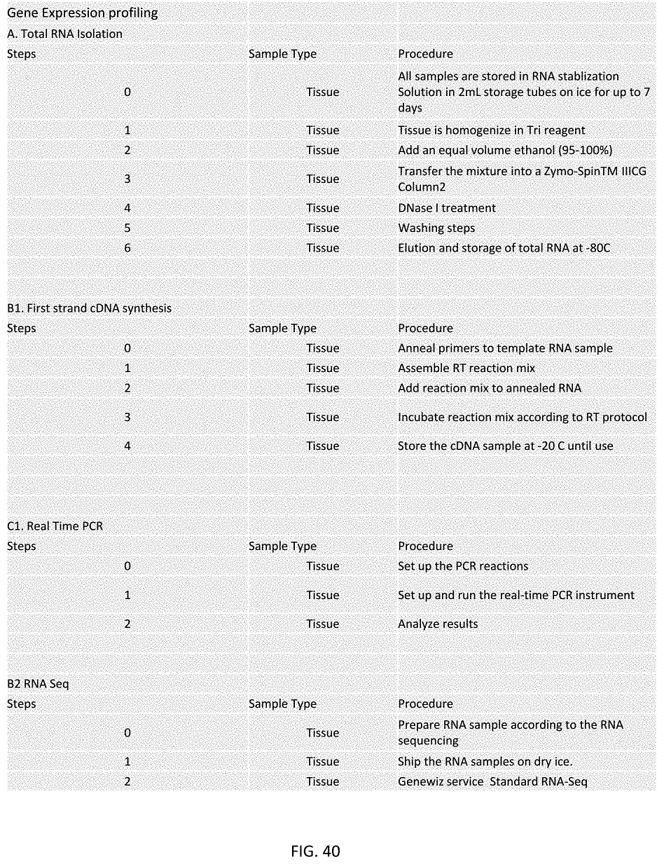

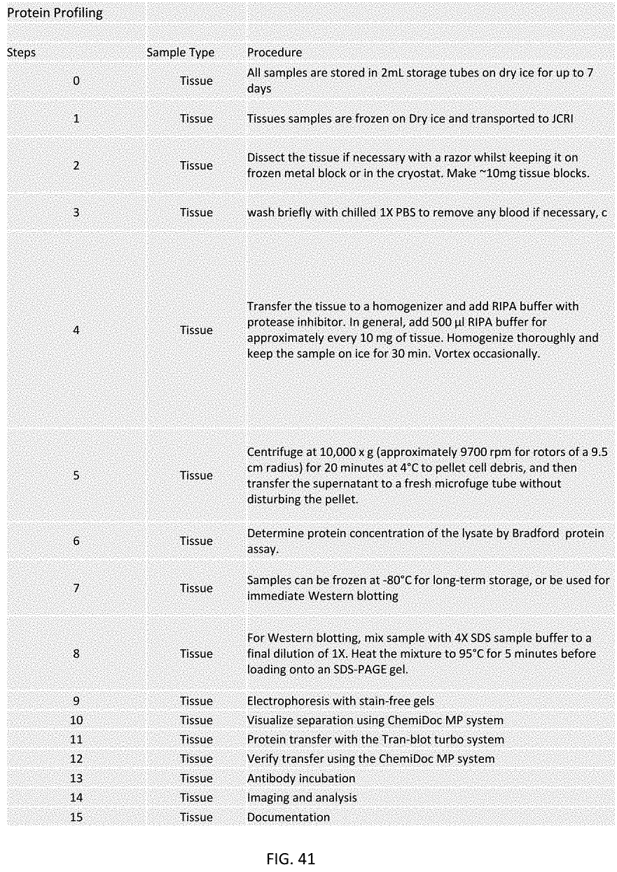

Translational molecular processing also was performed on resected cancer tissue and blood from the subject patients. FIG. 29 is a diagram showing translational molecular processing of blood samples taken from the subject patients. FIG. 30 is a diagram showing translational molecular processing of tissue samples taken from the subject patients. Methods for the profiling are outlined in FIGS. 31A-41.

The foregoing description of the preferred embodiment of the invention has been presented for purposes of illustration and description. It is not intended to be exhaustive or to limit the invention to the precise form disclosed, and modifications and variations are possible in light of the above teachings or may be acquired from practice of the invention. The embodiment was chosen and described in order to explain the principles of the invention and its practical application to enable one skilled in the art to utilize the invention in various embodiments as are suited to the particular use contemplated. It is intended that the scope of the invention be defined by the claims appended hereto, and their equivalents. The entirety of each of the aforementioned documents is incorporated by reference herein.

What is claimed is:

1. A method for treatment of solid cancer tumors comprising:
pre-operatively treating a patient having a solid cancer tumor with at least one of chemotherapy and radiation therapy;
surgically removing the solid cancer tumor from the patient; and
applying cold atmospheric plasma to the surgical margins surrounding the area in the patient from which the solid cancer tumor was removed.

2. A method for treatment of solid cancer tumors according to claim 1, wherein said step of pre-operatively treating a patient comprises treating the patient pre-operatively with both chemotherapy and radiation therapy.

3. A method for treatment of solid cancer tumors according to claim 1, further comprising:
post-operatively treating the patient having a solid cancer tumor with at least one of chemotherapy and radiation therapy.

4. A method for treatment of solid cancer tumors according to claim 3, wherein said step of post-operatively treating the patient comprises treating the patient pre-operatively with both chemotherapy and radiation therapy.

5. A method for treatment of solid cancer tumors according to claim 1, wherein said applying cold atmospheric plasma to the surgical margins comprises applying cold atmospheric plasma to the surgical margins for no more than 5 minutes.

6. A method for treatment of solid cancer tumors according to claim 1, wherein said applying cold atmospheric plasma to the surgical margins comprises applying cold atmospheric plasma to the surgical margins for no more than 3 minutes.

7. A method for treatment of solid cancer tumors according to claim 1, wherein said applying cold atmospheric plasma to the surgical margins comprises applying cold atmospheric plasma to the surgical margins for no more than 1 minute.

8. A method for treatment of solid cancer tumors according to claim 1, wherein said applying cold atmospheric plasma to the surgical margins comprises applying cold atmospheric plasma to the surgical margins at 120p.

9. A method for treatment of solid cancer tumors according to claim 1, wherein said applying cold atmospheric plasma to the surgical margins comprises applying cold atmospheric plasma to the surgical margins with a helium flow rate of 3 l/min.

10. A method for treatment of solid cancer tumors according to claim 1, further comprising:
intra-operatively treating the patient having a solid cancer tumor with at least one of chemotherapy and radiation therapy.

11. A method for treatment of solid cancer tumors according to claim 10, wherein said step of intra-operatively treating the patient comprises treating the patient intra-operatively with both chemotherapy and radiation therapy.

12. A method for treatment of solid cancer tumors according to claim 1, further comprising performing at least one of cytoreductive surgery and peritonectomy combined with hyperthermic intraperitoneal chemotherapy on the patient.
